(19) United States
(12) Patent Application Publication
(10) Pub. No.: US 2026/0103449 A1
(43) Pub. Date: Apr. 16, 2026

(54) PRODRUG ATHELIAPYRROLIDINE COMPOUND AND METHODS OF USE

(71) Applicant: UNIVERSITY OF FLORIDA RESEARCH FOUNDATION, INCORPORATED, Gainesville, FL (US)

(72) Inventors: Sandra LOESGEN, St. Augustine, FL (US); Annika JAGELS, SAINT AUGUSTINE, FL (US)

(21) Appl. No.: 19/362,990

(22) Filed: Oct. 20, 2025

Related U.S. Application Data
(63) Continuation-in-part of application No. PCT/US2024/024719, filed on Apr. 16, 2024.
(60) Provisional application No. 63/460,514, filed on Apr. 19, 2023.

(51) Int. Cl. C07D 295/13; A61K 31/40; A61K 31/7056; C07H 1/06; C07H 15/26

(52) U.S. Cl. CPC C07D 295/13; A61K 31/40; A61K 31/7056; C07H 1/06; C07H 15/26

(57) ABSTRACT

This disclosure relates to a prodrug form of a 5-HT_{2A} receptor antagonist atheliapyrrolidine compound and its use for producing 5-HT_{2A} receptor antagonism in a subject. The compound is useful for treatment of major depressive disorder, schizophrenia, obsessive-compulsive disorder, major depressive disorder, pervasive developmental disorders (autism), panic disorder, post-traumatic stress disorder, and the like.

### CROSS-REFERENCE TO RELATED APPLICATIONS

This application is a United States continuation-in-part application which claims priority from international application no. PCT/US2024/024719, filed 16 Apr. 2024, which claims the benefit of U.S. provisional application Ser. No. 63/460,514, filed 19 Apr. 2023. The entire contents of the aforementioned applications is hereby incorporated by reference as if fully set forth herein.

### GOVERNMENT FUNDING SUPPORT

This invention was made with government support under grant no. CH-2020110, awarded by the National Science Foundation. The government has certain rights in the invention.

### BACKGROUND

### 1. Field

The invention relates to the field of clinical chemistry and medicine. In general, the invention relates to a prodrug form of a 5-HT_{2A} receptor antagonist atheliapyrrolidine compound and its use for producing 5-HT_{2A} receptor antagonism in a subject. The compound is useful for treatment of major depressive disorder, schizophrenia, obsessive-compulsive disorder, major depressive disorder, pervasive developmental disorders (autism), panic disorder, post-traumatic stress disorder, and the like.

### 2. Background

### A. Introduction

The termite egg mimicking sclerotia (also known as “termite balls” (TMBs)) of the Basidiomycota fungus Athelia sp. (the teleomorph of Fibularhizoctonia species) are brown and of a similar size but different shape to termite eggs. They have been studied since their discovery in 1997 in nests of the termite Reticulitermes speratus in Japan. At that time, bioassay results suggested that the sclerotia functioned to protect termite eggs from putative pathogens and that chemical cues were involved in recognition. Later, other termite species such as R. flavipes and R. virginicus were found to harbor TMBs in the United States, along with R. speratus. Interestingly, Coptotermes formosanus, considered as one of the most aggressive and economically devastating termite species in the United States, has also been found to allow TMBs in their nests.

Only certain termite species can recognize sclerotia and care for them like their eggs, and only specific species of basidiomycota from the Athelia and Trechispora genera are associated with those lower termite nests. Specialization therefore exists despite horizontal transmission. Recently it was shown that A. termitophila is competitive against other wood-decay and most likely also entomopathogenic fungi at low temperatures, giving the termites a protective advantage in winter. In return, at warmer temperatures the termites provide a competitor-free habitat for A. termitophila, suggesting a seasonal mutualistic symbiosis. In contrast, higher termites of genus Macrotermes are well studied to cultivate fungus-gardens and associate preferentially with specific fungal species (Termitomyces).

Up to now, research has not been able to rule out the possibility that other supporting compounds are involved in termite egg recognition. Chemical studies have focused on termites and covered carbohydrate cues like 1,4-glycoside hydrolases, pheromones, common small volatile organic compounds, and a fungistatic (R-(−)-mellein, a dihydroisocoumarin derivative), which showed growth inhibition toward other intrusive, entomopathogenic fungi. However, no classic or state-of-the-art natural products chemistry, such as small molecules or putative secondary metabolites have been described for the part of the fungus in this unique ecological interaction.

### B. Selective 5-HT_{2A} Receptor Antagonists

Serotonin (5-hydroxytryptamine, 5-HT) activates 14 genetically-encoded 5-HT receptors in humans, most of which are G protein-coupled receptors (GPCRs). 5-HT_{2A} receptors (5-HT_{2A}R) have been well studied, and a numerous compounds have been found to be selective antagonists of this specific receptor to greater or lesser degrees. Serotonergic psychedelic compounds such as LSD, mescaline, and 2,5-dimethoxy-4-methyl-amphetamine act as agonists at this receptor (and others). The 5-HT_{2A}R has become one of the main targets for treatment of various aspects of psychosis such as schizophrenia, obsessive-compulsive disorder, major depressive disorder, pervasive developmental disorders (autism), panic disorder, post-traumatic stress disorder, and the like. Commonly used 5-HT_{2A}R antagonists include, but are not limited to clozapine, olanzapine, quetiapine, risperidone and asenapine, mirtazapine, mianserin, and the like.

### SUMMARY

There is a need in the art for new drugs and drug formulae for producing 5-HT_{2A} Receptor Antagonism in a patient. The invention described herein thus provides embodiments related to compounds and methods for achieving this.

In particular, the invention provides an atheliapyrrolidine prodrug according to the structure of Formula I:

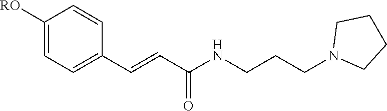

wherein R is a sugar, sugar alcohol, amino sugar, organic acid (e.g. acetic acid), amino acid,

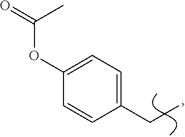

trigonelline, a purine, or a pyrimidine, with the proviso that R is not glucuronic acid. A further embodiment of the invention pertains to a method of producing 5-HT_{2A} receptor antagonism in a subject in need thereof. The method involves administering a therapeutically effective amount of atheliapyrrolidine A, atheliapyrrolidine A-GlcA, or an atheliapyrrolidine prodrug as described herein to the subject. In certain embodiments, the invention also provides a method of treating 5-HT_{2A} related disorder in a subject in need thereof. The method involves administering a therapeutically effective amount of atheliapyrrolidine A, atheliapyrrolidine A-GlcA, or an atheliapyrrolidine prodrug as described herein, or compositions containing the foregoing, to the subject. Another embodiment of the invention pertains to a pharmaceutically acceptable salt of atheliapyrrolidine A or atheliapyrrolidine A-GlcA. These and other embodiments are further described herein.
In particular embodiments, the present invention relates to an atheliapyrrolidine prodrug compound of Formula I:

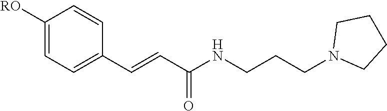

wherein R is a sugar, sugar alcohol, amino sugar, organic acid (e.g. acetic acid), amino acid,

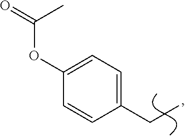

trigonelline, purine, or pyrimidine, with the proviso that R is not glucuronic acid, or a pharmaceutically acceptable salt thereof.

In certain embodiments, the invention provides a pharmaceutical composition comprising a pharmaceutically acceptable excipient and atheliapyrrolidine A, atheliapyrrolidine A-GlcA, or the atheliapyrrolidine prodrug of Formula I.

In additional embodiments, the invention relates to a method of producing 5-HT_{2A} receptor antagonism in a subject in need thereof, comprising administering a therapeutically effective amount of atheliapyrrolidine A, atheliapyrrolidine A-GlcA, or the atheliapyrrolidine prodrug of Formula I to the subject. In a specific embodiments, the method of producing 5-HT_{2A} receptor antagonism in a subject in need thereof, comprises administering a therapeutically effective amount of atheliapyrrolidine A-GlcA to the subject.

In certain embodiments, the invention provides a method of treating a subject in need thereof comprising administering to the subject a therapeutically effective amount of the pharmaceutical compositions disclosed herein, wherein the subject suffers from a 5-HT_{2A} related disorder. The 5-HT_{2A} related disorder preferably is a cardiovascular disorder, a psychiatric or neurological disorder, or pancreatitis. The cardiovascular disorder can be selected from the group consisting of platelet aggregation, coronary artery disease, myocardial infarction, transient ischemic attacks, stable angina, unstable angina, thrombotic stroke, a secondary ischemic event, atrial fibrillation, and thrombosis. The psychiatric or neurological disorder can be selected from the group consisting of alcohol and substance use disorders, anxiety disorders, panic disorder, agoraphobia and other specific phobias, social anxiety disorder, post-traumatic stress disorder, obsessive compulsive disorder, generalized anxiety disorder, bipolar disorder, sleep and wake disorders, depression, anorexia nervosa, binge eating disorder, bulimia nervosa, psychosis, schizophrenia, autism spectrum disorders, developmental disorders, and personality disorders.

### BRIEF DESCRIPTION OF THE DRAWINGS

Certain embodiments are illustrated by way of example, and not by way of limitation, in the figures of the accompanying drawings.

FIGS. 1A and 1B present the putative biosynthetic pathway for atheliapyrrolidine A.

FIG. 2 shows the total synthesis of atheliapyrrolidine A.

FIG. 3A is a QTOF-MS² spectrum of atheliapyrrolildine A Glc-A ([M+H]⁺ 451.2078, calc'd m/z 451.2075), collision-induced dissociation (25 eV), dashed lines show generated fragment ions with corresponding m/z, neutral loss 176.0324 indicates glucuronic acid moiety.

FIG. 3B shows the structure of atheliapyrrolidine A-GlcA ((2S,4S,6S)-3,4,5-trihydroxy-6-(4-((E)-3-oxo-3-((3-(pyrrolidin-1-yl) propyl)amino)prop-1-en-1-yl)phenoxy)tetrahydro-2H-pyran-2-carboxylic acid).

FIG. 4A and FIG. 4B show the COSY and HMBC correlations of atheliapyrrolidine A-GlcA and atheliapyrrolidine A, respectively.

FIG. 5 presents a qualitative analysis of atheliapyrrolidine A using mass spectrometric ion counts. Extracted ion chromatograms (EICs) for the m/z value for atheliapyrrolidine A found in extracts of 16 TMBs from laboratory cultivation (a), 4 termite eggs from the field (b), and 16 TMBs from the field (c). The isotopic pattern of atheliapyrrolidine is shown for the field sample of (c) are shown.

FIG. 6A presents the isotopic pattern of atheliapyrrolidine A-GlcA after feeding of D-glucose-d₆ (240 mg/L) and L-(+)-ornithine-d₇ (40 mg/L) in liquid culture after 25 days of cultivation. [M+H]⁺ 457.2447 (Δ ppm −1.1) corresponds to atheliapyrrolidine A-GlcA-d₆, [M+H]⁺; 451.2073 corresponds to the non-labeled atheliapyrrolidine A-GlcA.

FIG. 6B presents an MS² of isotopically labeled atheliapyrrolidine A-GlcA (CID 25 eV). Fragmentation indicates isotopically labeled aglycone atheliapyrrolidine A m/z 281.2129 (Δ ppm −0.71) and incorporation of do in the pyrrolidine moiety due to non-labeled fragments m/z 204.1021 and m/z 147.0442 (B).

FIG. 7A is a diode array detector (DAD) chromatogram (254 nm) of methanolic extract of an Athelia sp. TMB strain TB5 from liquid culture, supplemented with ¹³C₆-D-glucose (99%) and peptone, two weeks cultivation.

FIG. 7B shows +ESI scans of ¹³C-labeled metabolite (atheliapyrrolidine A-GlcA), two major products: m/z 457.2275 (C₁₆¹³C₆H₃₁N₂O₈⁺, Δppm −0.3) and m/z 466.2578 C₇¹³C₁₅H₃₁N₂O₈⁺, Δppm −0.03), (m/z 469.2405, C₂₀H₃₃N₆O₇⁺, Δppm −0.05, is a peptide derived from peptone).

FIG. 7C presents an MS² of m/z 457.2275 indicating ¹³C-labeled glucuronic acid (GlcA).

FIG. 7D presents an MS² of m/z 466.2578 showing ¹³C-labeled GlcA and p-coumaric acid moiety.

FIG. 8A shows results from a primary binding screening of atheliapyrrolidine A against 45 common receptors of neuroactive substances.

FIG. 8B shows results from a second primary binding screening assay of atheliapyrrolidine A against 45 common receptors of neuroactive substances.

FIG. 9A shows results from a secondary binding assay to the 5-HT_{2A} receptor for atheliapyrrolidine A.

FIG. 9B shows results from a secondary binding assay of the 5-HT_{2A} receptor for synthesized atheliapyrrolidine A (synthAtheliapyrrolidine A).

FIG. 9C shows the functional 5-HT_{2A} receptor TANGO antagonist assay results of atheliapyrrolidine A via β-arrestin signaling dose-response (response in relative luminescence units, RLU), (n=3), antagonist assay.

FIG. 9D is the functional 5-HT_{2A} receptor TANGO agonist assay for atheliapyrrolidine A.

FIG. 10A and FIG. 10B show results from secondary binding assays to the H3 and Sigma2 receptors, respectively, for atheliapyrrolidine A-GlcA.

FIG. 11A presents results from secondary binding assays of the 5-HT_{1E} receptor for the isolated natural product atheliapyrrolidine A.

FIG. 11B presents results from secondary binding assays of the DAT receptor for the isolated natural product atheliapyrrolidine A.

FIG. 11C presents results from secondary binding assays of the H3 receptor for the isolated natural product atheliapyrrolidine A.

FIG. 11D presents results from secondary binding assays of the Sigma2 receptor for the isolated natural product atheliapyrrolidine A.

FIG. 12 is a graph showing results for atheliapyrrolidine A stability studies with CD1mice plasma.

FIG. 13 is a graph showing results for atheliapyrrolidine A stability studies with simulated gastric fluid (SGF) and simulated intestinal fluid (SIF).

FIG. 14 is a graph showing results for atheliapyrrolidine A metabolic stability studies using CD1 male mice liver microsomes and mixed gender human liver microsomes.

### DETAILED DESCRIPTION

### 1. Definitions

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense. Unless defined otherwise, all technical and scientific terms used herein have the same meaning as commonly understood by one of ordinary skill in the art. Although various methods and materials similar or equivalent to those described herein can be used in the practice or testing of the present invention, suitable methods and materials are described below. However, the skilled artisan understands that the methods and materials used and described are examples and may not be the only ones suitable for use in the invention.

Moreover, as measurements are subject to inherent variability, any temperature, weight, volume, time interval, pH, salinity, molarity or molality, range, concentration and any other measurements, quantities or numerical expressions given herein are intended to be approximate and not exact or critical figures unless expressly stated to the contrary. Any numerical value inherently contains certain errors necessarily resulting from the standard deviation found in their respective testing measurements at the time of this writing. Notwithstanding that the numerical ranges and parameters setting forth the broad scope of the invention are approximations, the numerical values set forth in specific non-limiting examples are reported as precisely as possible.

Throughout this specification and the claims, unless the context requires otherwise, the word “comprise” and its variations, such as “comprises” and “comprising,” will be understood to imply the inclusion of a stated item, element or step or group of items, elements or steps but not the exclusion of any other item, element or step or group of items, elements or steps. Furthermore, the indefinite article “a” or “an” is meant to indicate one or more of the item, element or step modified by the article.

As used herein, the term “about” means plus or minus 20 percent of the recited value, so that, for example, “about 0.125” means 0.125±0.025, and “about 1.0” means 1.0±0.2. Furthermore, unless otherwise clear from the context, a numerical value presented herein has an implied precision given by the least significant digit. All ranges disclosed herein are to be understood to encompass any and all sub-ranges subsumed therein. For example, a range of “less than 10” can include any and all sub-ranges between (and including) the minimum value of zero and the maximum value of 10, that is, any and all sub-ranges having a minimum value of equal to or greater than zero and a maximum value of equal to or less than 10, e.g., 1 to 4.

As used herein, the term “5-HT_{2A} receptor antagonist” as used herein refers to a receptor antagonist that that blocks or dampens agonist- or partial agonist-mediated responses rather than provoking a biological response itself upon binding to a 5-HT2A receptor.

The terms “treatment” and “therapy”, as used in the present application, refer to a set of hygienic, pharmacological, surgical and/or physical means used with the intent to cure and/or alleviate a disease and/or symptoms with the goal of remediating the health problem. The terms “treatment” and “therapy” include preventive and curative methods, since both are directed to the maintenance and/or re-establishment of the health of an individual or animal. Regardless of the origin of the symptoms, disease and disability, the administration of a suitable medicament to alleviate and/or cure a health problem should be interpreted as a form of treatment or therapy within the context of this application. Treating includes, but is not limited to, administering a composition comprising one or more active agents to a subject using any known method for purposes such as curing, reversing, alleviating, reducing the severity of, inhibiting the progression of, or reducing the likelihood of a disease, disorder, or condition or one or more symptoms or manifestations of a disease, disorder or condition.

The term “psychiatric disorder” refers to a diagnosis by a mental health professional of a behavioral or mental pattern that may cause suffering or a poor ability to function in life. “Psychiatric disorders” may be persistent, relapsing and remitting, or occur as a single episode. In a preferred embodiment, the term “psychiatric disorder” refers to one or more disorders selected from the following: alcohol and substance use disorders, anxiety disorders, panic disorder, agoraphobia and other specific phobias, social anxiety disorder, post-traumatic stress disorder, obsessive compulsive disorder, generalized anxiety disorder, bipolar disorder, sleep and wake disorders, depression, anorexia nervosa, binge eating disorder, bulimia nervosa, psychosis, schizophrenia, autism spectrum disorders, developmental disorders, and personality disorders.

The term “active agent,” as used herein, refers to the component of a pharmaceutical product or pharmaceutical composition which is biologically active. In the present application, an active agent may comprise one or more compounds. The term does not encompass inactive ingredients such as pharmaceutically active carriers and/or diluents.

The term “co-administer” or other grammatical forms thereof, as used herein refers to the administration of an active agent before, concurrently, or after the administration of another active agent such that the biological effects of either agents overlap.

The term “neurological disorder” refers to any structural, biochemical and/or electrical abnormalities in the brain, spinal cord or other nerves. In a preferred embodiment, the term “neurological disorder” refers to one or more disorders selected from the following: acquired brain injury, ataxia, brain tumor, dementia, dystonia, epilepsy, temporal lobe epilepsy, pain associated with neurological disorders, headache disorders, functional and dissociative neurological symptoms, neuroinfections, meningitis, disorders associated with malnutrition, motor neuron disease, multi-system atrophy, multiple sclerosis, amyotrophic lateral sclerosis, mesial temporal lobe sclerosis, muscular dystrophy, myalgic encephalomyelitis, Parkinson's disease, progressive supranuclear palsy, cerebral palsy, Huntington's disease, Alzheimer's disease, fronto-temporal dementia, vascular dementia, dementia with Lewy bodies, corticobasal degeneration, Lyme encephalopathy, toxic encephalopathy, cognitive decline associated with aging, spina bifida, hydrocephalus, spinal injury, stroke, Tourette syndrome, and transverse myelitis.

The term “prevention”, as used in the present application, refers to a set of hygienic, pharmacological, surgical and/or physical means used to prevent the onset and/or development of a disease and/or symptoms. The term “prevention” encompasses prophylactic methods, since these are used to maintain the health of an animal or individual.

The term 5-HT2A related disorder refers to a psychological disorder, a neurological disorder, cardiovascular disorder, pancreatitis, or any other condition that is mediated by a 5-HT_{2A} receptor.

The term “cardiovascular disorder” refers to platelet aggregation, coronary artery disease, myocardial infarction, transient ischemic attacks, stable angina, unstable angina, thrombotic stroke, a secondary ischemic event, atrial fibrillation, and thrombosis.

The terms “subject,” “individual,” and “patient,” are used interchangeably herein to refer to an animal being treated with one or more enumerated compounds as taught herein, including, but not limited to, simians, humans, avians, felines, canines, equines, rodents, bovines, porcines, ovines, caprines, mammalian farm animals, mammalian sport animals, and mammalian pets. A suitable subject for the invention can be any animal, preferably a human, that is suspected of having, has been diagnosed as having, or is at risk of developing a disease that can be ameliorated, treated or prevented by administration of one or more enumerated compounds.

The terms “administering” or “administer” or “administration” as used herein with respect to an agent means providing the agent to a subject using any of the various methods or delivery systems for administering agents or pharmaceutical compositions known to those skilled in the art. Modes of administering include, but are not limited to oral administration, parenteral administration such as intravenous, subcutaneous, intramuscular or intraperitoneal injections, rectal administration by way of suppositories, transdermal administration, intraocular administration or administration by any route or method that delivers a therapeutically effective amount of the drug or composition to the cells or tissue to which it is targeted. Alternatively, routine experimentation will determine other acceptable routes of administration.

The term “enumerated compound(s)” as used herein encompass(es), for example, any 5-HT_{2A} receptor antagonist compound or activatable version thereof disclosed herein including any pharmaceutically acceptable salt or solvate thereof. Specific examples of compounds of the invention include those according to any chemical structure presented herein and any subgenera and/or species, or a pharmaceutically acceptable salt or solvate thereof. As used herein, the term “pharmaceutically acceptable salt” is intended to include nontoxic base addition salts. Suitable salts include those derived from organic and inorganic acids such as, without limitation, hydrochloric acid, hydrobromic acid, phosphoric acid, sulfuric acid, methanesulfonic acid, acetic acid, tartaric acid, lactic acid, sulfinic acid, citric acid, maleic acid, fumaric acid, sorbic acid, aconitic acid, salicylic acid, phthalic acid, and the like. The term “pharmaceutically acceptable salt” as used herein is also intended to include salts of acidic groups, such as a carboxylate, with such counterions as ammonium, alkali metal salts, particularly sodium or potassium, alkaline earth metal salts, particularly calcium or magnesium, and salts with suitable organic bases such as lower alkylamines (methylamine, ethylamine, cyclohexylamine, and the like) or with substituted lower alkylamines (e.g. hydroxyl-substituted alkylamines such as diethanolamine, triethanolamine or tris(hydroxymethyl)-aminomethane), or with bases such as piperidine or morpholine. Further examples of appropriate salts are described herein. Enumerated compound includes the disclosed structure or a stereoisomer thereof. The term “activatable version” refers to a form of a compound that includes a moiety that is removed upon administration wherein removal of the moiety converts the compound from an inactive version to active version. In specific examples, the enumerated compound is atheliapyrrolidine A (AP) or athelialpyrrolidine A-GlcA (APG). In another specific example, the enumerated compound is Formula I below:

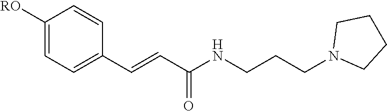

wherein R is a sugar, sugar alcohol, amino sugar, amino acid, purine, or pyrimidine, or

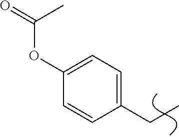

with the proviso that R is not glucuronic acid.

As used herein, “pharmaceutically acceptable carrier” or “pharmaceutically acceptable diluent” means any and all solvents, dispersion media, coatings, antibacterial and antifungal agents, isotonic and absorption delaying agents, compatible with pharmaceutical administration. The use of such media and agents for pharmaceutically active substances is well known in the art. Acceptable carriers, excipients, or stabilizers are nontoxic to recipients at the dosages and concentrations employed and, without limiting the scope of the present invention, include: additional buffering agents; preservatives; co-solvents; antioxidants, including ascorbic acid and methionine; chelating agents such as EDTA; metal complexes (e.g., Zn-protein complexes); biodegradable polymers, such as polyesters; salt-forming counterions, such as sodium, polyhydric sugar alcohols; amino acids, such as alanine, glycine, glutamine, asparagine, histidine, arginine, lysine, ornithine, leucine, 2-phenylalanine, glutamic acid, and threonine; organic sugars or sugar alcohols, such as lactitol, stachyose, mannose, sorbose, xylose, ribose, ribitol, myoinisitose, myoinisitol, galactose, galactitol, glycerol, cyclitols (e.g., inositol), polyethylene glycol; sulfur containing reducing agents, such as urea, glutathione, thioctic acid, sodium thioglycolate, thioglycerol, [alpha]-monothioglycerol, and sodium thio sulfate; low molecular weight proteins, such as human serum albumin, bovine serum albumin, gelatin, or other immunoglobulins; and hydrophilic polymers, such as polyvinylpyrrolidone. Other pharmaceutically acceptable carriers, excipients, or stabilizers, such as those described in Remington's Pharmaceutical Sciences 16th edition, Osol, A. Ed. (1980) may also be included.

The term “5-hydroxytryptamine receptor 2A (5-HT2A)” refers to a subtype of the 5-HT receptors that belongs to the serotonin receptor family and is a G protein-coupled receptor. The 5-HT2A receptor is found in humans and the receptor has been sequenced, characterized and the data have been deposited in the UniProtKB database under the accession number P28223.

The term “receptor antagonist” as used in the present application refers to a type of receptor ligand and/or drug that blocks or dampens agonist- or partial agonist-mediated responses rather than provoking a biological response itself upon binding to a receptor. The term “receptor agonist” refers to a type of receptor ligand and/or drug that activates the receptor to produce a full (full agonist) or partial (partial agonist) biological response. As used in the present application, the term “receptor antagonist” may also refer to a type of receptor ligand and/or drug that activates the receptor to produce a biological response that is opposed to that produced by a full or partial agonist. Although these compounds are technically known as “inverse agonists,” here we use the term “receptor antagonist” to encompass both antagonists and inverse agonists. The reason being that some reports in the scientific literature initially labeled a given compound as an “antagonist,” while subsequent more detailed studies have found the same compound to display inverse agonist activity. Both antagonists and inverse agonists effectively counteract the effects of agonists (full or partial).

A “therapeutically effective amount” refers to an amount which, when administered in a proper dosing regimen, is sufficient to reduce or ameliorate the severity, duration, or progression of the disorder being treated (e.g., neurological disorder), prevent the advancement of the disorder being treated (e.g., neurological disorder), cause the regression of the disorder being treated (e.g., neurological disorder), or enhance or improve the prophylactic or therapeutic effects(s) of another therapy. The full therapeutic effect does not necessarily occur by administration of one dose and may occur only after administration of a series of doses. Thus, a therapeutically effective amount may be administered in one or more administrations per day for successive days.

### 3. Exemplary Embodiments of the Invention

### A. Products and Synthesis

With the published draft genome sequence of Athelia (Fibularhizoctonia) sp. TMB strain TB5, it became possible to mine for certain biosynthetic gene clusters, for instance, the psilocybin cluster in Athelia. Some gene homologs were identified, but not biosynthetic gene clusters were found. In this study, other alkaloids in the fungus were discovered. A selective 5-HT_{2A} receptor antagonist fungal alkaloid was found and studied: atheliapyrrolidine A ((E)-3-(4-hydroxyphenyl)-N-(3-(pyrrolidin-1-yl) propyl) acrylamide) and atheliapyrrolidine A-GlcA (((2S,4S,6S)-3,4,5-trihydroxy-6-(4-((E)-3-oxo-3-((3-(pyrrolidin-1-yl) propyl)amino) prop-1-en-1-yl) phenoxy)tetrahydro-2H-pyran-2-carboxylic acid)).

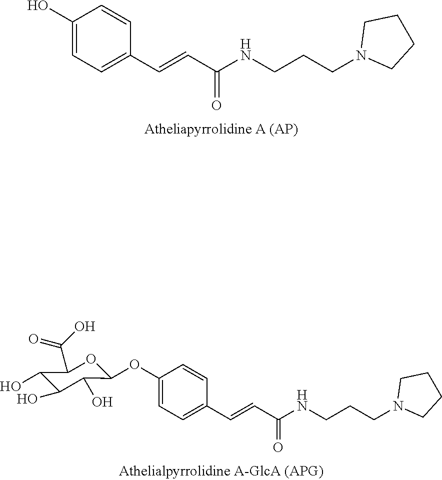 [SMILES: O=C(/C=C/c1ccc(O)cc1)NCCCN1CCCC1 ; O=C(/C=C/c1ccc(OC2OC(C(=O)O)C(O)C(O)C2O)cc1)NCCCN1CCCC1]

Atheliapyrrolidine A is the bioactive product. It is believed that the glucuronic acid is cleaved in vivo by 1,4-β-glucuronidase, which releases the bioactive alkaloid aglycone. Atheliapyrrolidine A: CAS No.: 1615712-49-6; molecular formula: C₁₆H₂₂N₂O; SMILES: OC(C═C1)=CC═C1/C═C/C(NCCCN2CCCC2)=O, MW: 274.4 g/mol; structure:

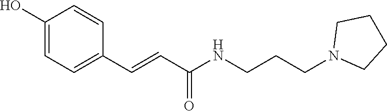 [SMILES: O=C(/C=C/c1ccc(O)cc1)NCCCN1CCCC1]

IUPAC names: (E)-3-(4-hydroxyphenyl)-N-(3-(pyrrolidin-1-yl)propyl)acrylamide (ChemDraw); (2E)-3-(4-hydroxyphenyl)-N-[3-(1-pyrrolidinyl) propyl]-2-propenamide (Chinese Patent below).

### Synthesis:

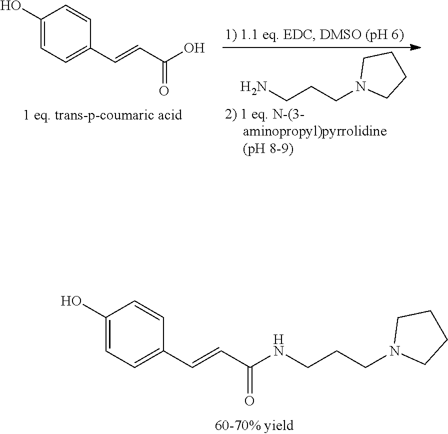 [SMILES: O=C(O)/C=C/c1ccc(O)cc1 ; NCCCN1CCCC1 ; O=C(/C=C/c1ccc(O)cc1)NCCCN1CCCC1]

Atheliapyrrolidine A-GlcA: molecular formula: C₂₂H₃₀N₂O₈; SMILES: O═C(NCCCN1CCCC1)/C═C/C2=CC═C(C═C2)O[C@@H]3O[C@H](C(O)═O)[C@@H](O)[C@H](O)[C@H]3O; MW: 450.5 g/mol; structure:

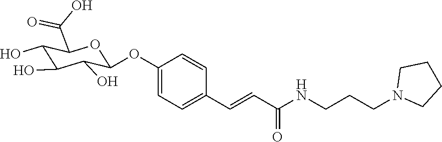 [SMILES: O=C(/C=C/c1ccc(OC2OC(C(=O)O)C(O)C(O)C2O)cc1)NCCCN1CCCC1]

Atheliapyrrolidine A-GlcA and atheliapyrrolidine A are unique to the metabolome of Athelia (Fibularhizoctonia) TMB strain TB5 and its teleomorph A. termitophila. In order to examine whether metabolites APG and AP are unique to the metabolome of Athelia (Fibularhizoctonia) sp. TMB strain TB5, its teleomorph (A. termatophila, athtmb2) or other Athelia species such as A. arachnoidea and A. epiphylla, fungal strains were grown under the same conditions, extracted and the compounds analyzed by LC-HRMS. Not surprisingly, A. termatophila, identified as the sexual reproductive stage (morph), also produces compounds APG and AP. Notably, neither compound was detected (lower than limit of detection) from extracts of A. arachnoidea and A. epiphylla.

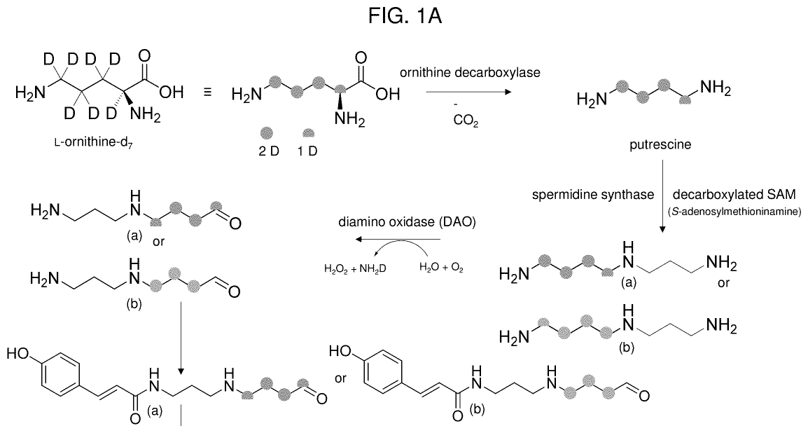

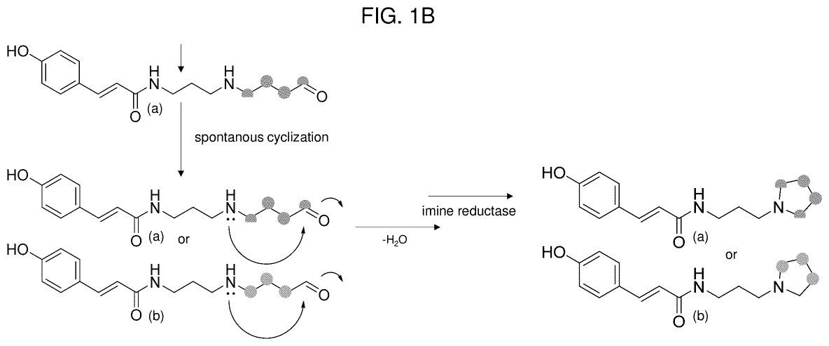

The putative biosynthetic pathway of AP is shown in FIG. 1. Liquid culture of Athelia (Fibularhizoctonia) sp. TMB strain TB5 was supplemented with L-ornithine-d₇ as indicated, ornithine decarboxylase leads to putrescine, followed by a spermidine synthase harnessing decarboxylated S-adenosylmethionine to generate spermidine (two possible versions ((a) or (b)). Diamino oxidase (DAO)/flavin-containing polyamine oxidase leads to corresponding amino aldehyde ((a) or (b)) which spontaneously cyclizes to give a pyrrolium intermediate (not shown), which is finally reduced via an imine reductase to generate AP ((a) or (b)).

Activatable Versions of Atheliapyrrolidine A (atheliapyrrolidine prodrugs). According to other embodiments, the enumerated compound is an activatable version of atheliapyrrolidine A. In certain embodiments the activatable version is provided in Formula I below:

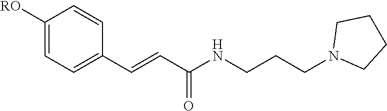

wherein R is a sugar, sugar alcohol, amino sugar, amino acid, purine, or pyrimidine, with the proviso that R is not glucuronic acid. In another embodiment, R is

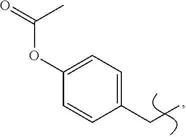

It is known that this protective group is cleaved in vivo by liver esterases. For example where R is

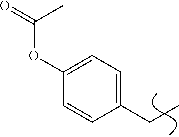

the following reaction would occur upon being subjected to a liver esterase:

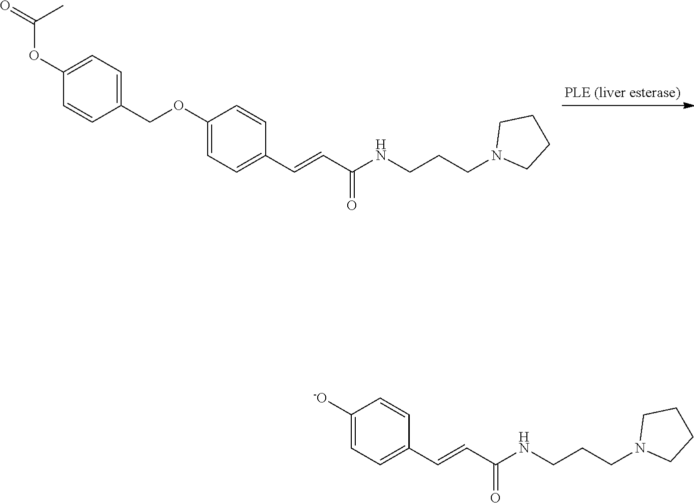

See Perez et al. ChemMedChem, 2013, 8(10): 1662-1667. R may also be an organic acid such as acetic acid. Those skilled in the art will appreciate that reference to R as a sugar, sugar alcohol, amino sugar, amino acid, organic acid

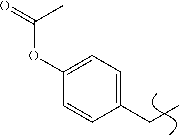

or purine or pyrimidine means that such compound forms an amine, ether or ester linkage. In an alternative embodiment, R may also be trigonelline as is described in U.S. Pat. No. 10,752,589, e.g., R is

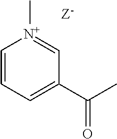

to form an ester linkage.

In a specific embodiment, the activatable version is synthesized as follows:

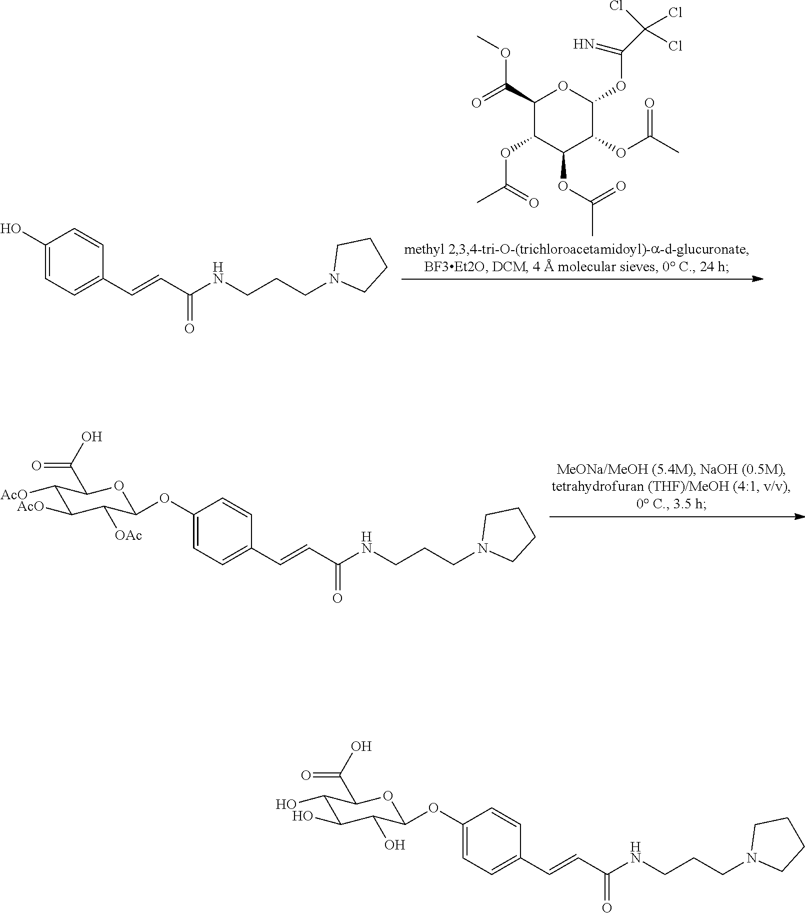 [SMILES: O=C(/C=C/c1ccc(O)cc1)NCCCN1CCCC1 ; COC(=O)[C@H]1O[C@H](OC(=N)C(Cl)(Cl)Cl)[C@H](OC(C)=O)[C@@H](OC(C)=O)[C@@H]1OC(C)=O ; CC(=O)OC1C(Oc2ccc(/C=C/C(=O)NCCCN3CCCC3)cc2)OC(C(=O)O)C(OC(C)=O)C1OC(C)=O ; O=C(/C=C/c1ccc(OC2OC(C(=O)O)C(O)C(O)C2O)cc1)NCCCN1CCCC1]

In the synthesis scheme above, the trichloroacetamidoyl donor can be swapped with any sugar, sugar alcohol, amino sugar, amino acid, purine, or pyrimidine. Alternatively, other syntheses start with the glucoside and then oxidize to the glucuronide: β-d-glucoside to β-d-glucuronide by 1) NaOCl, TEMPO, (If phenols are Bn protected and sugar OH are acetyl protected OAc, then second, reductive step with H₂/PD/C is needed). See Kajjout and Rolando, Tetrahedron, Volume 67, Issue 25, 24 Jun. 2011, Pages 4731-4741, which is incorporated by reference herein.

Glucuronidation of phenols is often performed using methyl-2,3,4-tri-O-acetyl-1-O-(trichloroacetimidoyl)-α-d-glucuronate, the coupling step being activated by Lewis acids, such as BF₃-etherate. More reactive reagents may be used like methyl-2,3,4-tri-O-acetyl-1-O-(trifluoroacetimidoyl)-α-d-glucuronate or 2,3,4-tri-O-acetyl-α-d-methyl glucuronopyranosyl-(N-phenyl)-2,2,2-trifluoroacetimidate in order to improve the yield. This acidic glucuronidation is usually performed on fully or partially protected phenols.

### B. Administration

An enumerated compound may be administered to an individual who suffers from one or more cardiovascular, psychiatric and/or neurological disorders and/or who is at risk of suffering from one or more psychiatric and/or neurological disorders.

In one embodiment, atheliapyrrolidine A, atheliapyrrolidine A-GlcA, or a compound described by formula (I) may be administered to an individual who is suffering from one or more psychiatric and/or neurological disorders and/or who is at risk of suffering from one or more psychiatric and/or neurological disorders. Conversely, in an alternative embodiment, a 5-HT2A receptor antagonist may be administered to an individual who is already being administered a compound described by formula (I) and who is suffering from one or more psychiatric and/or neurological disorders and/or who is at risk of suffering from one or more psychiatric and/or neurological disorders.

In a further embodiment, the enumerated compound is prepared for oral, sublingual, buccal, intranasal, intravenous, intramuscular, subcutaneous, rectal, transdermal, topical and/or inhalation-mediated administration routes, preferably oral, sublingual, inhalation-mediated and/or intranasal routes.

The pharmaceutical formulations of the present invention are prepared by methods well-known in the pharmaceutical arts. For example, the enumerated compounds are brought into association with a carrier and/or diluent, as a suspension or solution. Optionally, one or more accessory ingredients (e.g., buffers, flavoring agents, surface active agents, and the like) also are added. The choice of carrier is well within the prevue of the person of ordinary skill in the relevant art and is determined by the solubility and chemical nature of the compounds, chosen route of administration and standard pharmaceutical practice. These accessory ingredients and materials are well known in the art and include (1) fillers or extenders, such as starches, lactose, sucrose, glucose, mannitol, and silicic acid; (2) binders, such as carboxymethylcellulose, alginates, gelatin, polyvinyl pyrrolidone, hydroxypropylmethyl cellulose, sucrose and acacia; (3) humectants, such as glycerol; (4) disintegrating agents, such as agar-agar, calcium carbonate, potato or tapioca starch, alginic acid, certain silicates, sodium starch glycolate, cross-linked sodium carboxymethyl cellulose and sodium carbonate; (5) solution retarding agents, such as paraffin; (6) absorption accelerators, such as quaternary ammonium compounds; (7) wetting agents, such as cetyl alcohol and glycerol monostearate; (8) absorbents, such as kaolin and bentonite clay; (9) lubricants, such as talc, calcium stearate, magnesium stearate, solid polyethylene glycols, and sodium lauryl sulfate; (10) suspending agents, such as ethoxylated isostearyl alcohols, polyoxyethylene sorbitol and sorbitan esters, microcrystalline cellulose, aluminum metahydroxide, bentonite, agar-agar and tragacanth; (11) buffering agents; (12) excipients, such as lactose, milk sugars, polyethylene glycols, animal and vegetable fats, oils, waxes, paraffins, cocoa butter, starches, tragacanth, cellulose derivatives, polyethylene glycol, silicones, bentonites, silicic acid, talc, salicylate, zinc oxide, aluminum hydroxide, calcium silicates, and polyamide powder; (13) inert diluents, such as water or other solvents; (14) preservatives; (15) surface-active agents; (16) dispersing agents; (17) control-release or absorption-delaying agents, such as hydroxypropylmethyl cellulose, other polymer matrices, biodegradable polymers, liposomes, microspheres, aluminum monosterate, gelatin, and waxes; (18) opacifying agents; (19) adjuvants; (20) wetting agents; (21) emulsifying and suspending agents; (22), solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan; (23) propellants, such as chlorofluorohydrocarbons and volatile unsubstituted hydrocarbons, such as butane and propane; (24) antioxidants; (25) agents which render the formulation isotonic with the blood of the intended recipient, such as sugars and sodium chloride; (26) thickening agents; (27) coating materials, such as lecithin; and (28) sweetening, flavoring, coloring, perfuming and preservative agents. Each such ingredient or material must be “acceptable” in the sense of being compatible with the other ingredients of the formulation and not injurious to the subject. Ingredients and materials suitable for a selected dosage form and intended route of administration are well known in the art, and acceptable ingredients and materials for a chosen dosage form and method of administration may be determined using ordinary skill in the art.

For administration to a suitable subject, preferably to a human patient suffering from or suspected of suffering from 5-HT_{2A} related disorder, the compounds described here are prepared according to methods known in the art into suitable formulations for any route of administration and suitable doses. Suitable subjects for administration and treatment can be any mammal, including rats, mice, dogs, cats, farm animals such as cattle, sheep, horses and the like or any mammal.

### C. Dosing and Dosage Forms

The appropriate dose of an enumerated compound depends upon a number of factors within the ken of the ordinarily skilled physician, veterinarian, or researcher for example, the identity, size, and condition of the subject or sample being treated, further depending upon the route by which the composition is to be administered, the frequency of administration, the severity of the disease, and the effect which the practitioner desires the an active agent to have. Furthermore, appropriate doses of an active agent depend upon the potency with respect to the expression or activity to be modulated. Such appropriate doses may be determined using the assays described herein or which are convenient to the practitioner and know in the art. When one or more of these active agents are to be administered to an animal (e.g., a human), a relatively low dose may be prescribed at first, with the dose subsequently increased until an appropriate response is obtained.

In addition, the specific dose level for any particular subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

Dosages and regimens for administration are determined by the person of skill, including physicians. Administration of compositions, including the compounds described here, can be performed a single time, or repeated at intervals, such as by continuous infusion or repeated oral doses, over a period of time, four times daily, twice daily, daily, every other day, weekly, monthly, or any interval to be determined by the skilled artisan based on the subject involved. Treatment can involve administration over a period of one day only, a week, a month, several months, years, or over a lifetime. Regimens and duration can vary according to any system known in the art, as is known to the skilled person.

Pharmaceutical compositions suitable for oral administration may be in the form of capsules, cachets, pills, tablets, powders, granules, a solution or a suspension in an aqueous or non-aqueous liquid, an oil-in-water or water-in-oil liquid emulsion, an elixir or syrup, a pastille, a bolus, an electuary or a paste. These formulations may be prepared by methods known in the art, e.g., by means of conventional pan-coating, mixing, granulation or lyophilization processes.

Solid dosage forms for oral administration (capsules, tablets, pills, dragees, powders, granules and the like) may be prepared, e.g., by mixing the active ingredient(s) with one or more pharmaceutically acceptable carriers and, optionally, one or more fillers, extenders, binders, humectants, disintegrating agents, solution retarding agents, absorption accelerators, wetting agents, absorbents, lubricants, and/or coloring agents. Solid compositions of a similar type maybe employed as fillers in soft and hard-filled gelatin capsules using a suitable excipient. A tablet may be made by compression or molding, optionally with one or more accessory ingredients. Compressed tablets may be prepared using a suitable binder, lubricant, inert diluent, preservative, disintegrant, surface-active or dispersing agent. Molded tablets may be made by molding in a suitable machine. The tablets, and other solid dosage forms, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein. They may be sterilized by, for example, filtration through a bacteria-retaining filter. These compositions may also optionally contain opacifying agents and may be of a composition such that they release the active ingredient only, or preferentially, in a certain portion of the gastrointestinal tract, optionally, in a delayed manner. The active ingredient can also be in microencapsulated form.

Liquid dosage forms for oral administration include pharmaceutically acceptable emulsions, microemulsions, solutions, suspensions, syrups and elixirs. The liquid dosage forms may contain suitable inert diluents commonly used in the art. Besides inert diluents, the oral compositions may also include adjuvants, such as wetting agents, emulsifying and suspending agents, sweetening, flavoring, coloring, perfuming and preservative agents. Suspensions may contain suspending agents.

Pharmaceutical compositions for rectal or vaginal administration may be presented as a suppository, which may be prepared by mixing one or more active ingredient(s) with one or more suitable nonirritating carriers which are solid at room temperature, but liquid at body temperature and, therefore, will melt in the rectum or vaginal cavity and release the active compound. Pharmaceutical compositions which are suitable for vaginal administration also include pessaries, tampons, creams, gels, pastes, foams or spray formulations containing such pharmaceutically acceptable carriers as are known in the art to be appropriate.

Dosage forms for the topical or transdermal administration include powders, sprays, ointments, pastes, creams, lotions, gels, solutions, patches, drops and inhalants. The active agent(s)/compound(s) may be mixed under sterile conditions with a suitable pharmaceutically acceptable carrier. The ointments, pastes, creams and gels may contain excipients. Powders and sprays may contain excipients and propellants.

Pharmaceutical compositions suitable for parenteral administrations comprise one or more agent(s)/compound(s) in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain suitable antioxidants, buffers, solutes which render the formulation isotonic with the blood of the intended recipient, or suspending or thickening agents. Proper fluidity can be maintained, for example, by the use of coating materials, by the maintenance of the required particle size in the case of dispersions, and by the use of surfactants. These compositions may also contain suitable adjuvants, such as wetting agents, emulsifying agents and dispersing agents. It may also be desirable to include isotonic agents. In addition, prolonged absorption of the injectable pharmaceutical form may be brought about by the inclusion of agents which delay absorption.

In some cases, in order to prolong the effect of a drug (e.g., pharmaceutical formulation), it is desirable to slow its absorption from subcutaneous or intramuscular injection. This may be accomplished by the use of a liquid suspension of crystalline or amorphous material having poor water solubility.

The rate of absorption of the active agent/drug then depends upon its rate of dissolution which, in turn, may depend upon crystal size and crystalline form. Alternatively, delayed absorption of a parenterally-administered agent/drug may be accomplished by dissolving or suspending the active agent/drug in an oil vehicle. Injectable depot forms may be made by forming microencapsule matrices of the active ingredient in biodegradable polymers. Depending on the ratio of the active ingredient to polymer, and the nature of the particular polymer employed, the rate of active ingredient release can be controlled. Depot injectable formulations are also prepared by entrapping the drug in liposomes or microemulsions which are compatible with body tissue. The injectable materials can be sterilized for example, by filtration through a bacterial-retaining filter.

The formulations may be presented in unit-dose or multi-dose sealed containers, for example, ampules and vials, and may be stored in a lyophilized condition requiring only the addition of the sterile liquid carrier, for example water for injection, immediately prior to use. Extemporaneous injection solutions and suspensions may be prepared from sterile powders, granules and tablets of the type described above.

When active agents are used in combinations, the dose of each agent is expected to be approximately the same as, or less than, an effective amount of either alone. For example, each pharmaceutically active ingredient can be administered in doses that are about 20% to about 80% of the dose in which each ingredient would be administered alone.

The two (or more) agents can be administered more or less simultaneously, i.e., concomitantly (e.g., within about 0 to about 5 minutes of each other, preferably within about a minute apart), or they can be administered at different times. For example, the compositions can be formulated in a unit dosage form, each dosage containing both active ingredients. The term “unit dosage form” refers to physically discrete units suitable as unitary dosages for human subjects and other animals, each unit containing a predetermined quantity of active material calculated to produce the desired prophylactic or therapeutic effect over the course of a treatment period, in association with the required pharmaceutical carrier.

### D. Salts

Salts of the enumerated compounds disclosed herein include acid addition salts formed with inorganic acids such as hydrochloric acid, hydrobromic acid, sulfuric acid, nitric acid, phosphoric acid, and the like; or with organic acids such as acetic acid, propionic acid, hexanoic acid, heptanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 0-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, p-chlorobenzenesulfonic acid, 2-naphthalenesulfonic acid, p-toluenesulfonic acid, camphorsulfonic acid, 4-methylbicyclo[2.2.2]oct-2-ene-1-carboxylic acid, glucoheptonic acid, 4,4′-methylenebis(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid and the like. Salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like.

### 4. EXAMPLES

This invention is not limited to the particular processes, compositions, or methodologies described, as these may vary. The terminology used in the description is for the purpose of describing the particular versions or embodiments only, and is not intended to limit the scope of the present invention which will be limited only by the appended claims. Although any methods and materials similar or equivalent to those described herein can be used in the practice or testing of embodiments of the present invention, the preferred methods, devices, and materials are now described. All publications mentioned herein, are incorporated by reference in their entirety; nothing herein is to be construed as an admission that the invention is not entitled to antedate such disclosure by virtue of prior invention.

### Example 1: General Methods

### A. General Experimental Procedures

1D and 2D NMR spectra were recorded at 500 MHz for ¹H NMR and 150 MHz for ¹³C NMR at 298 K on an Agilent VNMRS instrument. Solvent peaks were used as internal references: DMSO-d₆ (δ_{H} 2.50; δ_{C} 39.5), D₂O (δ_{H} 4.80).

LC-DAD-HRMS experiments were conducted on an Agilent™ 1290 Infinity II series UPLC coupled to an Agilent™ 6546 QTOF mass spectrometer with an electrospray ionization (ESI) source (Agilent Technologies™). Chromatography was performed using a ZIC®-HILIC column (150× 2.1 mm, 3.5 μm, Merck™ KGaA), oven temperature was set to 40° C. and the sample injection volume was 1 μL. A binary gradient consisting of MeCN (eluent A) and water (eluent B), both+0.1% FA, at a constant flow rate of 400 μL/min was used. The gradient was applied as following: 0-2 min, 90% A; 10.00 min, 40% A; 13.00 min, 40% A; 13.01 min, 90% A; 18.00 min 90% A. For data acquisition and subsequent qualitative analysis, MassHunter™ software (Agilent Technologies™) was used.

All parameters regarding the high resolution mass spectrometry, QTOF, are provided here: UPLC-HRMS experiments were conducted on an Agilent™ 1290 Infinity II series UPLC coupled to an Agilent™ 6546 QTOF mass spectrometer with an electrospray ionization (ESI) source (Agilent Technologies™). Chromatography was performed using a ZIC-HILIC® column (150×2.1 mm, 2.6, Merck™), oven temperature was set to 40° C. and the sample injection volume was 1 μL.

A binary gradient consisting of water (eluent A) and MeCN (eluent B) at a constant flow rate of 300 μL/min was used. The gradient was applied as following: 0-2.00 min, 60% A; 10.00 min 60% A; 13.00 min 60% A; 13.1 min, 10% A. For data acquisition and subsequent qualitative and quantitative analysis, MassHunter™ software (Agilent Technologies™) was used. All parameters regarding the QTOF are listed in Table 1, below (Agilent™ 6546 QTOF (Agilent Technologies™).

TABLE 1
Applied settings for the high-resolution
mass spectrometric analyses

| Ion Source | Dual AJS ESI | |
|---|---|---|
| Polarity | Positive | |
| Gas temp (° C.) | 350 | |
| Gas flow (L/min) | 11 | |
| Nebulizer (psig) | 40 | |
| SheathGasTemp | 375 | |
| SheathGasFlow | 12 | |
| VCap | 3000 | V |
| Nozzle Voltage | 0 | V |
| Fragmentor | 130 | |
| Skimmer1 | 45 | |
| OctopoleRFPeak | 750 | |
| Reference masses | m/z 121.0509, 922.0098 | |
| MS abs. threshold | 200 | |
| MS/MS abs. threshold | 5 | |
| Diverter valve | 0-1 min | (waste) |
| MS min-max range (m/z) | 100-800 | |
| MS scan rate (spectra/s) | 1.50 | |
| MS/MS min-max range (m/z) | 50-800 | |
| MS/MS scan rate (spectra/s) | 1.50 | |
| Isolation width MS/MS | Medium | (~4 amu) |
| Collision energy (CID) | 25.00 | eV |

| | |
|---|---|
| Max precursers/cycle, threshold (Abs) | 2, 200 |
| Isotope model | Peptides |
| Active exclusion enabled | No |
| Sort precursors | By abundance only |
| Selected charges | 1, 2, unknown |
| Data storage mode | Centroid |

LC-LRMS experiments were conducted on an Agilent™ 1100 series UPLC coupled to an Agilent™ 6130 mass spectrometer with an electrospray ionization (API_ES) source (AgilentTechnologies™). Injection volume was 10 μL and column oven temperature maintained at 30° C. For data acquisition and subsequent qualitative and quantitative analysis, Chemstation™ software (Agilent Technologies™) was used. See below for analytical parameters: Drying gas 12.00 (L/min); Nebulizer pressure (40 psig); Drying gas temp (350° C.), Capillary voltage 4000V; Peak width 0.1 min; Cycle time 1.09 sec/cycle; Mode: scan; Scan data storage: condensed; Polarity: positive; DAD: 210, 254, 300, 360 nm.

Chromatography was performed using a Kinetex® C18 column (150×4.6 mm, 5u, Phenomenex™), and the sample injection volume was 10 μL. A binary gradient consisting of water (eluent A) and MeCN (eluent B) (both+0.05% formic acid (FA) at a constant flow rate of 800 μL/min was used. The gradient was applied as following: 0-1.00 min, 95% A; 10.00 min 70% A; 10.10 min 5% A; 15.00 min 5% A; 15.10 min 95% A; 20.00 min 95% A.

### B. Fungal Strain and Culture Conditions

Sclerotia formation and isolation of atheliapyrrolidine A (solid medium): the fungal strain Athelia sp. TMB strain TB5 was obtained in July 2017 from a Reticulitermes flavipes colony at the Denison Bioreserve (Granville, OH, USA). The sclerotia were lyophilized and maintained at 4° C. before seeding on agar plates. Optimal sclerotia formation was achieved on malt extract-peptone agar (30 g/L CRITERION™ Hardy Diagnostics, VWR; 5 g/L peptone (Fisher Scientific™; 15 g/L agar, VWR; pH 5, no adjustment; autoclaved at 121° C. for 15 min; 100×15 mm petri dishes (Fisher Scientific™). Agar plates were inoculated with a single sclerotium in the center and incubated for 21 days at 26° C.

### C. Determination of Dry Biomass for the Biosynthesis of Atheliapyrrolidine A-GlcA (Liquid Medium) and Isotope Labeling.

125 mL Erlenmeyer flasks were filled with 50 mL liquid medium. The standard medium was as described above, malt extract-peptone medium. In order to monitor biosynthesis, modifications were the following, with consistent concentrations of 167 mM C-source (Glu) and 50 mM N-source unless stated otherwise (autoclaved separately to avoid Maillard reaction), prepared in triplicates:
D-glucose (Fisher Scientific™)+L-alanine (Fisher Scientific™)
D-glucose+ammonium chloride (Fisher Scientific™)
D-glucose+L-arginine (Fisher Scientific™)
D-glucose+L-aspartic acid (Fisher Scientific™)
D-glucose+L-asparagine (Fisher Scientific™)
D-glucose+ (sodium) L-glutamate monohydrate (Fisher Scientific™)
D-glucose+L-glutamine (Sigma-Aldrich™)
D-glucose+L-glycine (VWR™)
D-glucose+L-histidine (Fisher Scientific™)
D-glucose+L-isoleucine (Sigma-Aldrich™)
D-glucose+L-lysine (Fisher Scientific™)
D-glucose+L-methionine (Sigma-Aldrich™)
D-glucose+L-(+)-ornithine hydrochloride (Fisher Scientific™)
D-glucose+L-phenylalanine (Fisher Scientific™)
D-glucose+L-proline (Fisher Scientific™)
D-glucose+D-proline (Fisher Scientific™)
D-glucose+L-tryptophan (Fisher Scientific™)
   Isotope feeding:
750 mg/L U-¹³C6-D-glucose (Cambridge Isotope Laboratories™)+peptone 3 g/L 6-¹³C-D-glucose+10 g/L peptone
240 mg/L D-glucose+40 mg/L L-ornithine-2,3,3,4,4,5,5-d₇ hydrochloride (LGC standards, Toronto Research Chemicals™)
1,2-¹³C₂-acetate, 15 g/L D-glucose, 12 g/L (Cambridge Isotope Laboratories™)+5 g/L peptone

After autoclavation (121° C. for 15 min) flasks were inoculated with a 2-3 sclerotia and incubated at RT for 4 weeks (shaking, 120 rpm).

### D. Extraction and Isolation

Isolation of atheliapyrrolidine A: after 28 days of incubation, sclerotia were carefully scratched off from 80 agar plates and extracted with 80% MeCN+0.1% formic acid (FA) by ultra-sonication for 30 min (3×). The extracts were combined and concentrated in vacuo to obtain organic extract and the aqueous residue was lyophilized. Afterward, the residue was resuspended in H₂O and atheliapyrrolidine A was purified by semi-preparative HPLC-DAD (Agilent™ 1100) at 300 nm with a MeCN (A)/H₂O (B) gradient (both solvents+0.1% FA) linearly increasing from 5-30% A in 15 min on a Kinetex® C₁₈ column (150×10 mm, 5 μm, Phenomenex™), and a flow rate of 3.8 mL/min. 8 mg were yielded as a brownish amorphous powder.

Isolation of atheliapyrrolidine A-GlcA (1): extraction and isolation were analogous to atheliapyrroldine A, but extraction solvent was alkaline MeOH (pH 12) and the solvents for semi-preparative HPLC-UV contained no modifier (0.1% FA). 8 mg of pale-yellow amorphous powder was obtained.

Synthesis of atheliapyrrolidine A (2): Initially, to optimize and test successful reaction conditions, a micro-scale synthesis was carried out. Trans-p-coumaric acid (98%, TCI America™, Fisher Scientific™, 1 eq., 1.5 mg, 9.15 mmol,) and the crosslinker agent N-(3-dimethylaminopropyl)-N′-ethylcarbodiimide hydrochloride (EDC*HCl, Sigma-Aldrich™, 1.1 eq., 2.0 mg, 10.42 mmol) were dissolved in 1 mL DMSO, stirred in a vial for 25 min at r.t., before, N-(3-aminopropyl) pyrrolidine (98%, Acros Organics, Fisher Scientific™, 1 eq., 1.2 μL, 9.15 mmol) was added to the solution and stirred at r.t. overnight. The next day the reaction was monitored by LC-DAD-ESI-MS and the reaction yield determined according to the UV signal at 300 nm (50-60%). Then, a larger approach was conducted with 7.5 mg trans-p-coumaric acid (7.62 mmol, 1 eq.) in 6 mL dry DMF and 10 mg EDC*HCl (8.68 mmol, 1.1 eq.), stirred for 25 min at r.t. followed by the addition of 6 μL N-(3-aminopropyl) pyrrolidine (7.81 mmol, 1 eq.) and stirred at r.t. overnight. The reaction mixture was then pre-cleaned via solid phase extraction (Hypersep™ C₁₈ cartridge, 5 g bed weight, 25 mL, Thermo Fisher Scientific™) to remove the DMF and other polar by-products. The cartridge was equilibrated with MeCN and H₂O, the reaction mixture loaded, and the cartridge washed with H₂O, before eluting with 100% MeCN. The elute fraction was evaporated in vacuo and resuspended in H₂O for final purification via semi-preparative HPLC-DAD analogous as described above for the isolation of atheliapyrrolidine A 4 mg of the final product was obtained as pale-yellow powder (53%).

### E. Physical Data of Isolated and Synthesized Compounds

Atheliapyrrolidine A-GlcA (1): pale yellow amorphous powder, HRMS (ESI) m/z 451.2078, [M+H]⁺ calcd for C₂₂H₃₀N₂O₈, m/z 451.2075; 1H and ¹³C NMR, c.f. Table 5. UV/Vis (MeCN): λₘₐₓ=300 nm (c.f. SI). Atheliapyrrolidine A (2, natural product): brownish amorphous powder, HRMS (ESI) m/z 275.1744, [M+H]⁺ calcd for C₁₆H₂₂N₂O₂, m/z 275.1754; ¹H and ¹³C NMR, c.f. Table 5. UV/Vis (MeCN): λₘₐₓ=300 nm (c.f. SI).

Atheliapyrrolidine A (2, synthetic product): pale yellow amorphous powder, HRMS (ESI) m/z 275.1748, [M+H]⁺ calcd for C₁₆H₂₂N₂O₂, m/z 275.1754; UV/Vis (MeCN): λₘₐₓ=300 nm (c.f. SI); ¹H NMR (500 MHz, DMSO-d₆) δ 1.65 (p, J=7.3 Hz, 2H, H-2″″), 1.71 (bs, 4H, H-3′, H-4′), 2.51 (overlapped, 2H, H-3′″), 2.55 (bs, 4H, H-2′, H-5′), 3.18 (q, 2H, H-1′″), 6.39 (d, J=15.8 Hz, 1H, H-2″), 6.79 (d, J=8.6 Hz, 2H, H-3, H-5), 7.29 (d, J=15.8 Hz, 2H, H-3″), 7.37 (d, J=8.7 Hz, 2H, H-2, H-6), 8.04 (s, 1H, NH-1′), 8.37 (bs, 1H, NH); ¹³C NMR (150 MHZ, DMSO-d₆) δ 23.0 (CH₂, C-3′, C-4′), 28.1 (CH₂, C-2′″), 36.9 (CH₂, C-1′″), 53.0 (CH₂, C-3′″), 53.4 (CH₂, C-2′, C-5′), 115.8 (CH, C-3, C-5), 118.6 (CH, C-2″), 125.7 (C, C-1), 129.1 (CH, C-2, C-6), 138.6 (CH, C-3″), 159.1 (C, C-4), 165.4 (C, C-1″).

### F. Biological Assays

Colon cancer cells (HCT-116) were purchased from the American Type Culture Collection (ATCC, Manassas, VA). Dulbecco's modified Eagle's medium (DMEM), phosphate buffer saline (PBS), trypsin/EDTA (0.25%/2.21 mM) and penicillin/streptomycin solution were obtained from Fisher Scientific™. Fetal bovine serum (FBS) was obtained from R&D systems. Cells were cultivated in DMEM with 10% (v/v) fetal bovine serum, penicillin (100 U/mL), and streptomycin (100 μg/mL). The cell lines were incubated at 37° C., 5% CO₂. Cells were plated into a 96-well plate (7000 cells/well). Cell viability was determined by measuring the reduction of the tetrazolium salt MTT ((3-(4,5-dimethylthiazolyl-2)-2,5-diphenyltetrazolium bromide, VWR™) by metabolically active cells. After 24 hours of treatment, MTT (5 mg/mL in PBS) was added to each well at a final concentration of 0.5 mg/mL. The plates were incubated for 2 hours at 37° C. The medium was removed, cells were lysed, and the purple formazan product was solubilized by the addition of 50 μL of DMSO. Absorbance was measured at 550 nm with a microplate reader (Synergy HTX, Biotek™). Metabolic activity of vehicle-treated cells (0.5% DMSO unless otherwise stated) was defined as 100% cell growth. Etoposide (250 μM) was used as a positive control.

For antibacterial activity in cell-based assays, established protocols were followed (CLSI etc. ref.). Gram-positive bacteria, including Enterococcus faecium (ATCC 49032), Staphylococcus aureus (ATCC 25923), methicillin-resistant Staphylococcus aureus (ATCC BAA-41), multidrug-resistant Staphylococcus aureus (ATCC BAA-44) were tested. Candida albicans (ATCC 90027) and Cryptococcus neoformans (ATCC 14116) were deployed as pathogenic yeasts. Vancomycin, kanamycin, and amphotericin B were used as positive controls. DMSO served as negative control. For single-dose assay, pure compounds were tested at 125 μg/mL. Microbial growth rates (optical density) were measured after 16 hours by absorbance at 620 nm using a Biotek Synergy™ 96-well plate reader.

### G. PDSP Screening

All bioassay data reported below was generously provided by the National Institute of Mental Health's Psychoactive Drug Screening Program, Contract #HHSN-271-2018-00023-C (NIMH PDSP). The NIMH PDSP is directed by Bryan L. Roth at the University of North Carolina at Chapel Hill and Project Officer Jamie Driscoll at NIMH, Bethesda MD, USA.

Primary and secondary binding screens, as well as secondary functional assays were carried out according to the PDSP assay book for full protocols, reference pdf. Briefly, in primary binding assays, compounds are usually tested at a single concentration (10 μM) and in quadruplicate in 96-well plates. Compounds showing a minimum of 50% inhibition at 10 μM are tagged for secondary radioligand binding assays to determine equilibrium binding affinity at specific targets. In secondary binding assays, selected compounds are usually tested at 11 concentrations (0.1, 0.3, 1, 3, 10, 30, 100, 300 nM, 1, 3, 10 μM) and in triplicate (3 sets of 96-well plates). Secondary binding assays were run in triplicate using standard radio ligands. Secondary functional assays were run in triplicate against positive controls in TANGO assays.

Statistical comparisons and p-values *** p≤0.001; ** p≤0.01 were determined by an one-way analysis of variance (one-way ANOVA) and a Tukey's post hoc test.

### H. Additional Studies

Analytical Methods for metabolic stability in mice and human liver microsomes are described below (Examples 10-12 including LC-MS/MS methods, CD1 mouse plasma stability, Simulated Gastric Fluid (SGF, pH 1.2) and Simulated Intestinal Fluid (SIF, pH 6.8) Stability).

The analytical method is for UPLC-MS/MS (MRM scan) using a Waters Xevo TQ-S Micro & Waters Acquity I Class UPLC instrument.

TABLE 2
HPLC Parameters

| | |
|---|---|
| Mobile phase A | 0.1% formic acid in water |
| Mobile phase B | Acetonitrile |
| Flow rate | 0.35 mL/min |
| Run time | 3.0 minutes |
| Flow | Gradient |
| Column | Acquity UOLC BEH C18 μm, 2.1 mm × 50 mm |
| Column temperature | 50° C. |
| Sample temperature | 10° C. |
| Injection volume | 2 μL |
| Weak wash | 1:1:2 ACN:MeOH:IPA:water + 0.1% formic acid |
| Strong wash | 1:1:1:1 ACN:MeOH:IPA:water + 0.1% formic acid |
| Wash volumes | Weak: 800 μL; Strong: 400 μL |

TABLE 3
Mass Spectrometer Parameters

| | Ion transition | Cone | Collision |
|---|---|---|---|
| Compounds | (m/z) | (V) | energy (V) |
| Atheliapyrrolidine A | 275.05 > 203.94 | 12 | 18 |
| (qualifier) | | | |
| Atheliapyrrolidine A | 275.05 > 146.89 | 12 | 28 |
| (qualifier) | | | |
| Phenacetin (IS) | 180.12 > 110.03 | 34 | 20 |

Capillary voltage, 3.0 kV; desolvation gas flow, 1000 L/hour; cone gas flow, 40 L/hour; source temperature, 150° C.

TABLE 4
UPLC Gradient

| Time (minutes) | % A | Curve |
|---|---|---|
| Initial | 95 | Initial |
| 0.4 | 95 | 6 |
| 1.6 | 20 | 6 |
| 2.4 | 20 | 6 |
| 2.5 | 95 | 6 |
| 3.0 | 95 | 6 |

### Example 2: Synthesis and Elucidation of Chemical Structures

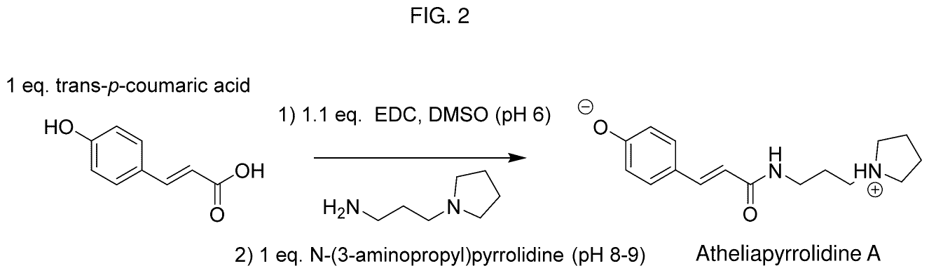

The total synthesis of atheliapyrrolidine A (56%) from 1 equivalent (eq.) trans-p-coumaric acid and 1) 2 eq. N-(3-Dimethylaminopropyl)-N′-ethylcarbodiimide hydrochloride (EDC) in DSMO at pH 6, 25 minutes, room temperature, followed by 2) 1 eq. N-(3-aminopropyl) pyrrolidine under alkaline conditions (pH 8-9), overnight, room temperature, is shown in FIG. 2.

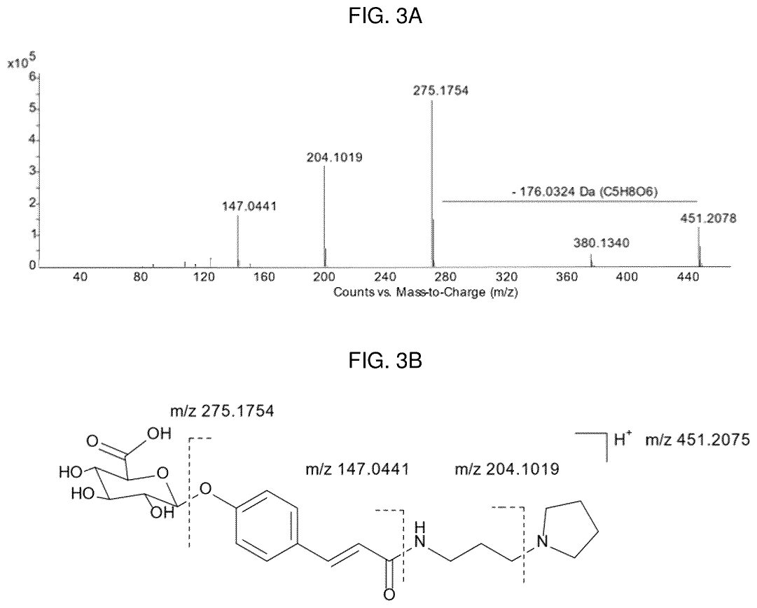

FIG. 3A is a QTOF-MS² spectrum of 1 ([M+H]⁺ 451.2078, calcd m/z 451.2075), collision-induced dissociation (25 eV), dashed lines show generated fragment ions with corresponding m/z, neutral loss 176.0324 indicates glucuronic acid moiety.

FIG. 3B shows the structure of atheliapyrrolidine A-GlcA.

### Example 3: NMR

NMR was conducted as described in Example 1. NMR data of atheliapyrridolidine A-Glc in D₂O and atheliapyrridolidine A in DMSO-d₆ (recorded at 500 MHz for ¹H and 150 MHz for ¹³C, δ in ppm, * signal overlapped) are presented in Table 5.

TABLE 5
NMR Data.

| Posi- | Atheliapyrrolidine A-GlcA | | Posi- | Atheliapyrrolidine A | |
|---|---|---|---|---|---|
| tion | δC | δH (J in Hz) | tion | δC | δH (J in Hz) |
| 2 | 76.2, CH | 3.92, d (9.3) | 1 | 125.8, C | |
| 3 | 71.7, CH | 3.63, m* | 2 | 129.1, CH | 7.37, d (8.6) |
| 4 | 75.3, CH | 3.64, m* | 3 | 115.7, CH | 6.78, d (8.4) |
| 5 | 72.7, C | 3.64, m* | 4 | 158.9, C | |
| 6 | 99.7, CH | 5.18, d (7.0) | 5 | 115.7, CH | 6.78, d (8.4) |
| 7 | 175.3, C | | 6 | 129.1, CH | 7.37, d (8.6) |
| 1′ | 158.0, C | | 1′ | NH+ | |
| 2′ | 116.8, CH | 7.62, d (9.2) | 2′ | 53.2, CH₂ | 2.70 (bs) |
| 3′ | 129.7, CH | 7.17, d (8.9) | 3′ | 22.9, CH₂ | 1.75 (bs) |
| 4′ | 129.3, C | | 4′ | 22.9, CH₂ | 1.75 (bs) |
| 5′ | 129.7, CH | 7.17, d (8.9) | 5′ | 53.2, CH₂ | 2.70 (bs) |
| 6′ | 116.8, CH | 7.62, d (9.2) | 1″ | 165.4, C | |
| 2″ | 26.0, CH₂ | 1.96, m* | 2″ | 118.6, CH | 6.38, d (15.7) |
| 3″ | 52.5, CH₂ | 3.08, t (6.2) | 3″ | 138.6, CH | 7.31, d (15.7) |
| 4″ | 53.9, CH₂ | 3.21, m* | 1″′ | 36.7, CH₂ | 3.19 (bs) |
| 5″ | 22.6, CH₂ | 2.01, m* | 2″′ | 27.5, CH₂ | 1.67 (bs) |
| 1″′ | 140.6, CH | 7.50, d (15.4) | 3″′ | 52.7, CH₂ | 2.65 (bs) |
| 2″′ | 118.6, CH | 6.54, d (15.7) | | | |
| 3″′ | 169.1, C | | | | |
| 1′v | 36.7, CH₂ | 3.39, t (6.2) | | | |
| 2′v | 26.0, CH₂ | 1.96, m* | | | |
| 3′v | 52.5, CH₂ | 3.08, t (6.2) | | | |

### Example 4: Selected COSY and HMBC Correlations of Atheliapyrrolidine A-GlcA and Atheliapyrrolidine A

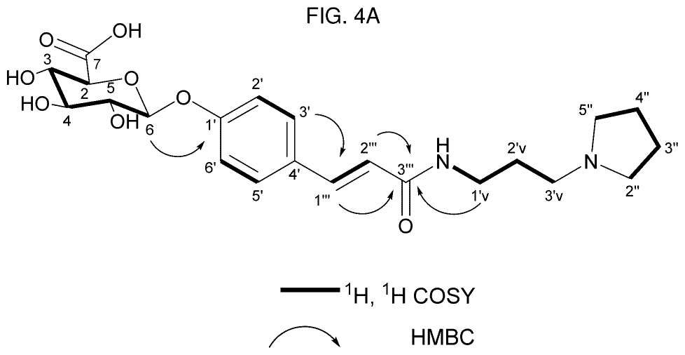

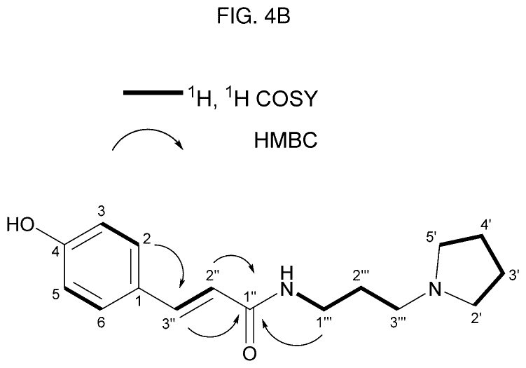

FIG. 4A and FIG. 4B show the 2D-NMR (COSY and HMBC) correlations of atheliapyrrolidine A-GlcA and atheliapyrrolidine A, respectively. The correlations prove the connectivity of spin systems in the molecule and verify the structure.

### Example 5: Qualitative Analysis of TMBs and Termite Eggs from the Field & Quantitation of AP and APG Per TMB

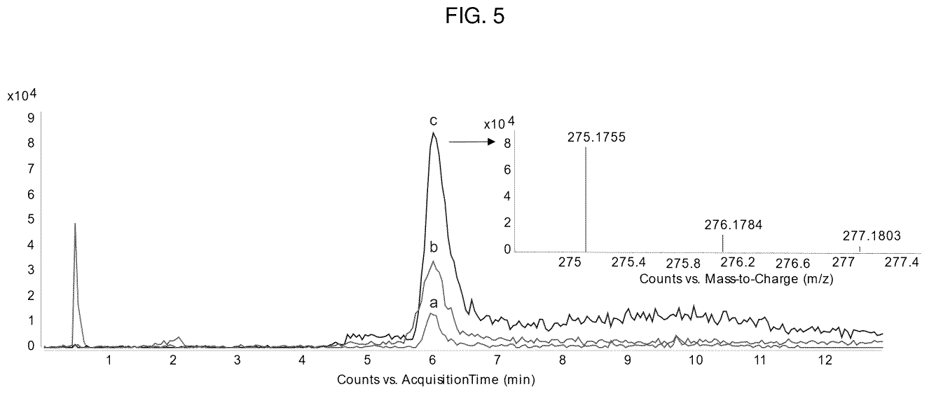

Qualitative and quantitative analysis for metabolites (AP and APG) was performed as described in Example 1. FIG. 5 presents an overlay of extracted ion chromatograms (EICs) of AP for extracts of 16 TMBs from laboratory cultivation (see line a), 4 termite eggs from the field (see line b), and 16 TMBs from the field (see line c). The inset shows the isotopic pattern of AP for the field sample of line c. Low-resolution mass spectrometer settings are listed elsewhere herein.

### Example 6: Biosynthesis & Putative BGCs/Genes/Loci, Isotope Feeding (d₇-Ornithine and ¹³C-Glucose), Other N-Sources

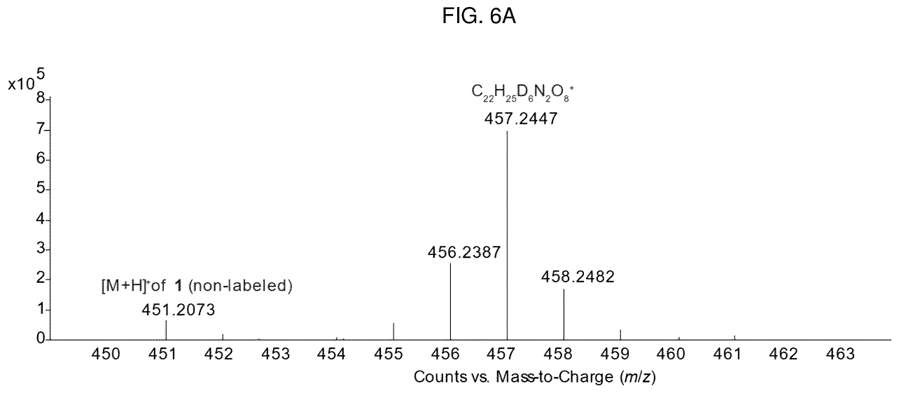

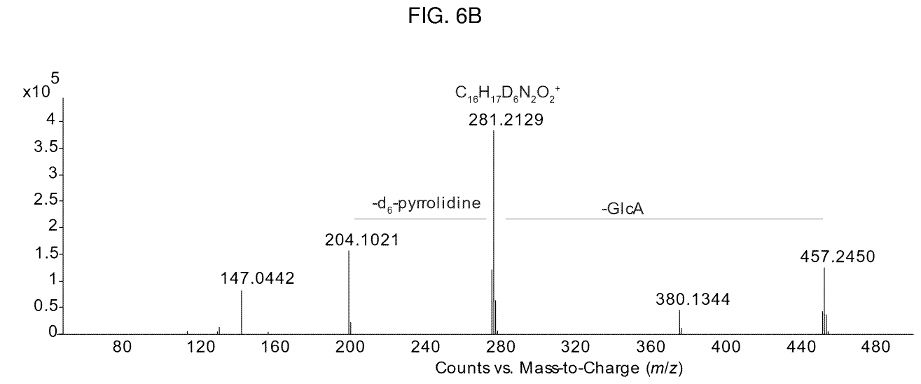

The isotopic pattern of APG was obtained as described in Example 1. Results are shown in FIG. 6A and FIG. 6B, FIG. 6A shows the isotopic pattern of APG after feeding of D-glucose (240 mg/L) and L-(+)-ornithine-d₇ (40 mg/L) in liquid culture after 25 days of cultivation, [M+H]⁺ 457.2447 (4 ppm-1.1) shows 1 incorporated d₆), [M+H]⁺ 451.2073 non-labeled APG. FIG. 6B shows the MS² of isotopically labeled 1 (CID 25 eV). Fragmentation indicates isotopically labeled aglycone 2 m/z 281.2129 (Δ ppm-0.71) and incorporation of do in the pyrrolidine moiety due to non-labeled fragments m/z 204.1021 and m/z 147.0442.

### Example 7: DAD Chromatogram of an Athelia sp. TMB Strain TB5

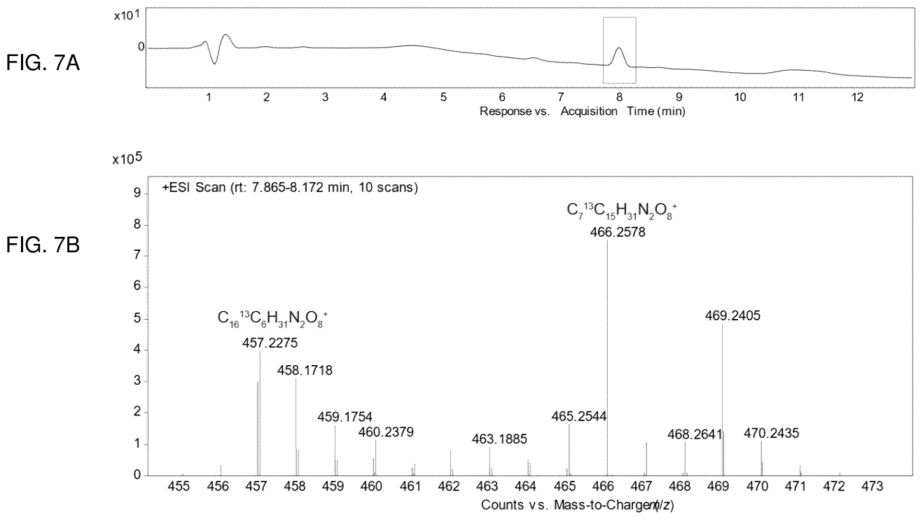

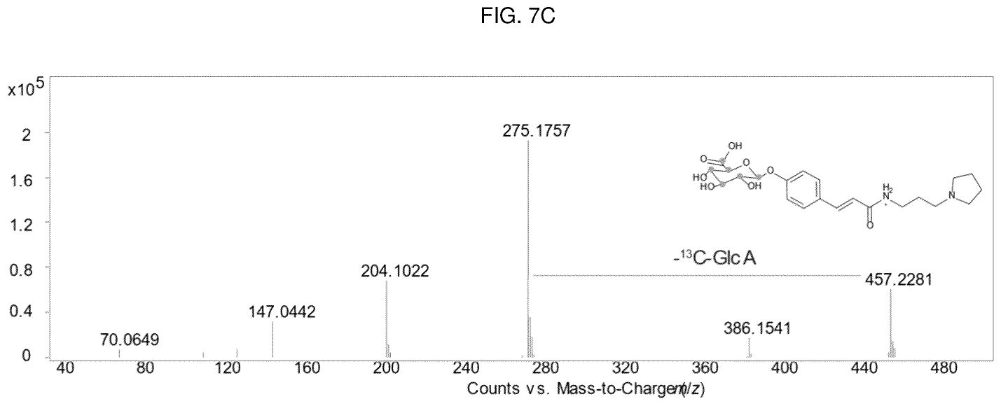

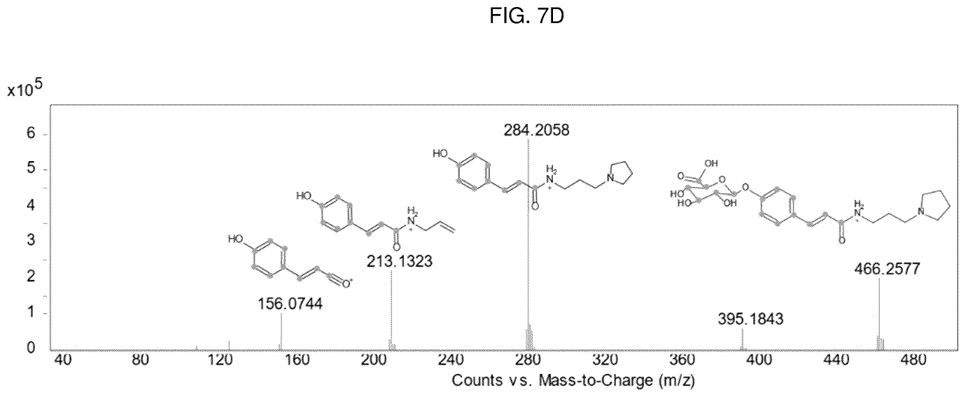

The following methods were used. FIG. 7A presents a DAD chromatogram (254 nm) of a methanolic extract of an Athelia sp. TMB strain TB5 from liquid culture, supplemented with ¹³C₆-D-glucose (99%) and peptone, two weeks of cultivation, FIG. 7B presents an +ESI scans of ¹³C-labeled metabolite APG. Two major products are shown: m/z 457.2275 (C₁₆¹³C₆H₃₁N₂O₈⁺, Δppm −0.3) and m/z 466.2578 C₇¹³C₁₅H₃₁N₂O₈⁺, Δppm −0.03), (m/z 469.2405, C₂₀H₃₃N₆O₇⁺. Δppm −0.05, peptide derived from peptone). FIG. 7C is an MS² of m/z 457.2275 indicating ¹³C-labeled glucuronic acid (GlcA). FIG. 7D is an MS² of m/z 466.2578 showing ¹³C-labeled GlcA and p-coumaric acid moiety.

### Example 8: PDSP Screening

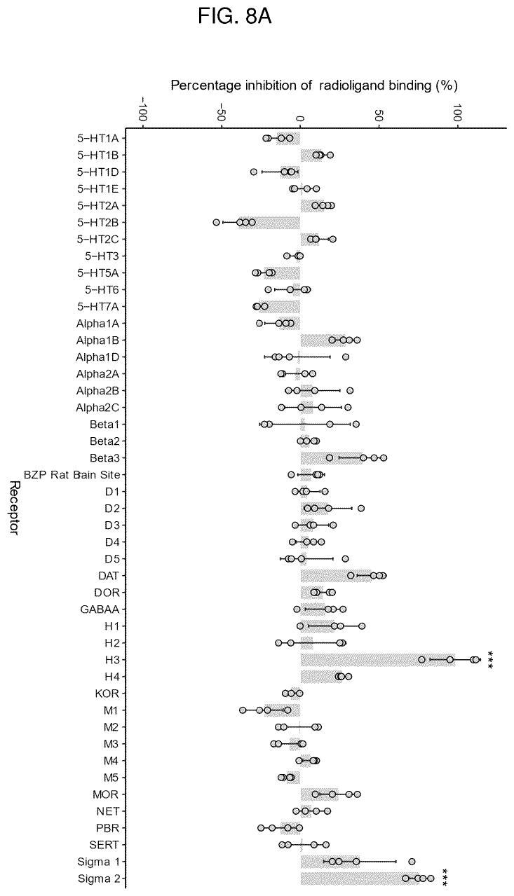

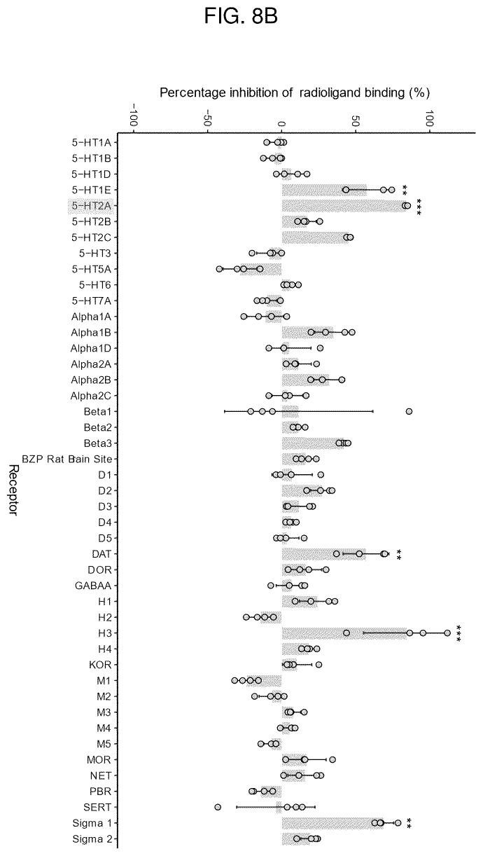

Primary binding screening of APG against 45 different common receptors of neuroactive substances was performed as described in Example 1. Results are shown in FIG. 8A. The values indicate % inhibition of radioligand binding by 10 μM of APG (n=4); *** p≤0.001, ** p≤0.01, significance compared to receptors with a mean below 30%, one-way ANOVA with post hoc Tukey's test. Error bars indicate standard deviations. Receptors showing a mean≥50% inhibition and *** p≤0.001 underwent secondary binding screening.

A second primary binding screening assay of AP was performed against the same 45 receptors, using the same methods. Results are shown in FIG. 8B. The values indicate % inhibition of radioligand binding by 10 μM of AP (n=4); *** p≤0.001, ** p≤0.01, significance compared to receptors with a mean below 30%, one-way ANOVA with post hoc Tukey's test. Error bars indicate standard deviations. Receptors showing a mean≥50% inhibition and *** p≤0.001, ** p≤0.01 underwent secondary binding screening.

### Example 9: Secondary Binding and Functional Assay Results

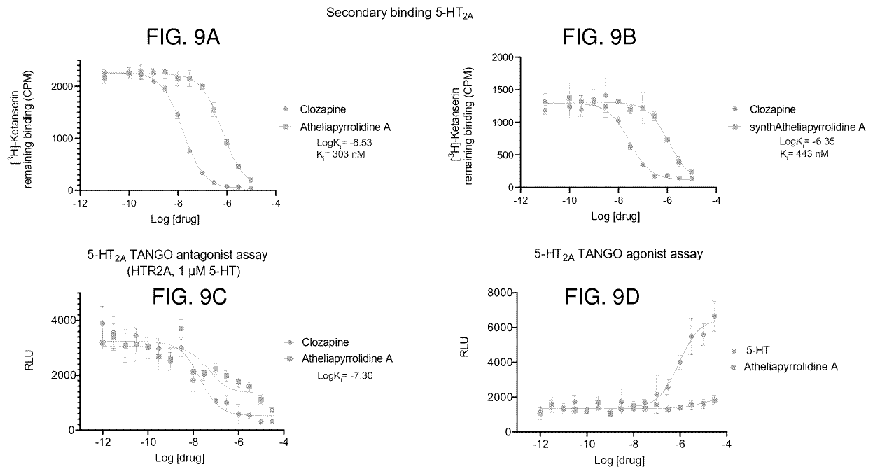

FIG. 9 shows secondary binding assays for AP (A) and the synthesized synthAtheliapyrrolidine A (B) on the 5-HT_{2A} receptor. Functional assays for AP acting on the 5-HT2A receptor, antagonist assay (C), agonist assay (D), via an in vitro TANGO β-arrestin signaling dose-response curve (response in relative luminescence units, RLU), (n=3).

The Tango GPCR assay is a reporter assay in which a transcription factor attached to the receptor is cleaved off by a protease-tagged β-arrestin, which leads to the expression of a reporter that creates a luminescence readout upon substrate addition. Atheliapyrrolidine A (AP) exhibits antagonistic activity similar to the positive control clozapine, but no agonist properties (see FIG. 9C and FIG. 9D).

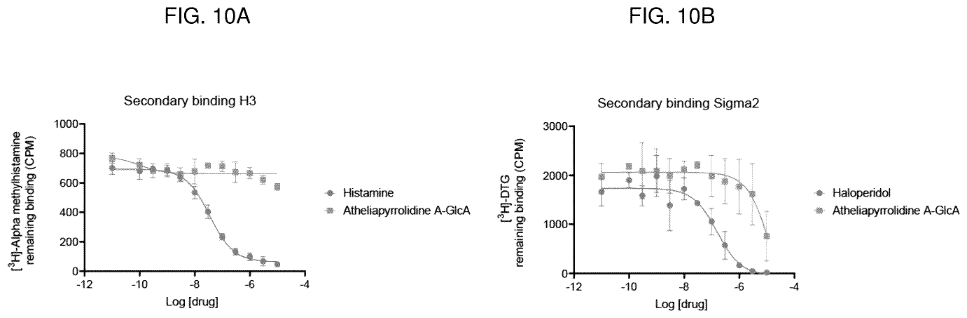

FIG. 10 shows secondary binding assays for APG to the H3 (A) and Sigma2 (B) receptors, Log Kᵢ=≥5. Although APG presented selectivity to the H₃ and Sigma2 receptors, the secondary binding assay does not show a dose-depending binding at relevant concentrations.

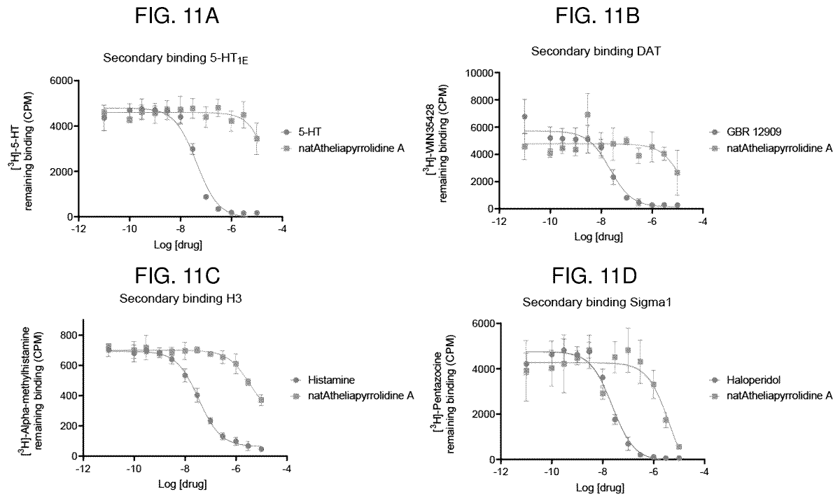

FIG. 11 shows secondary binding assays for AP to the 5-HT_{1E} (A), DAT (B), H3 (C); and Sigma2 (D) receptors, Log Kᵢ=≥−5. Although AP showed selectivity to the 5-HT_{1E}, DAT, H3, and Sigma2 receptors in FIG. 8B, the secondary binding assay does not reveal a dose-depending binding at relevant concentrations.

### Example 10: Plasma Stability

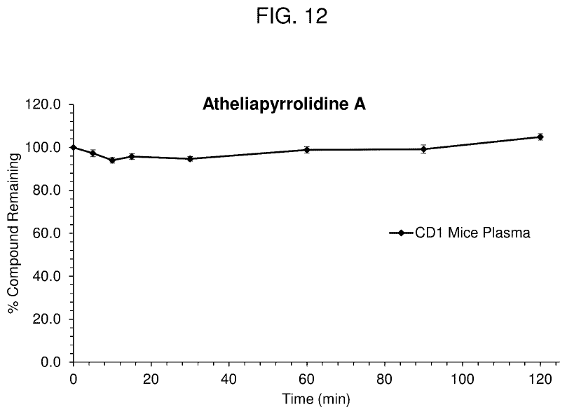

Plasma Stability studies were performed at 1 μM concentration in CD1 mice plasma (N=3). Compound was incubated with CD1 plasma for 2 hours in an incubator/shaker at 37.0° C. and 125 RPM. Twenty microliter samples were withdrawn at 0, 5, 10, 15, 30, 60, 90, and 120 minutes and mixed with 100 μl of methanol containing 0.05% formic acid and 20 ng/ml phenacetin (internal standard) followed by filtration centrifugation for 5 minutes at 2000 rpm using Solvinert™ 0.45 μm PTFE filter plates. Two microliters of the filtrate were injected into UPLC-MS/MS for analysis. Atheliapyrrolidine A was found to be stable for 2 hours in CD1 mice plasma. See results in FIG. 12.

### Example 11: SGF and SIF Stabiliity

SGF and SIF Stability studies were performed at 1 μM concentration in simulated gastric fluid (SGF, pH 1.2) and simulated intestinal fluid (SIF, pH 6.8) prepared as per USP specifications.

For the gastric fluid tests, 500 mg of sodium chloride and 800 mg of purified pepsin were dissolved in the SGF (derived from porcine stomach mucosa, with an activity of 800 to 2500 units per mg of protein, in 1.75 mL of 12.1 N hydrochloric acid and sufficient water to make 250 mL). Note that pepsin activity is described in the Food Chemicals Codex specifications under General Tests and Assays. This test solution has a pH of about 1.2.

For the intestinal fluid tests, 1.7 g of monobasic potassium phosphate was dissolved in 100 ml of water, mixed, and then 20 mL of 0.2 N sodium hydroxide and 150 mL of water was added. Pancreatin (2.5 g) was added and mixed, and the pH the resulting solution adjusted with either 0.2 N sodium hydroxide or 0.2 N hydrochloric acid to a pH of 6.8±0.1.

Compound was incubated with SGF or SIF for 2 hours in an incubator/shaker at 37° C. and 125 RPM (N=3 each). Twenty microliter samples were withdrawn at 0, 5, 10, 15, 30, 60, 90, and 120 minutes and mixed with 100 μL of methanol containing 0.05% formic acid and phenacetin (20 ng/ml) as internal standard followed by filtration centrifugation for 5 minutes at 2000 rpm using Solvinert™ 0.45 μm PTFE filter plates. Two microliters of the filtrate were injected to UPLC-MS/MS for analysis.

The analysis was performed using UPLC-MS/MS (MRM scan) on a Waters™ Xevo TQ-S Micro & Waters Acquity I Class UPLC. The parameters were as shown above under general methods, and below.
Mobile Phase: A: 0.1% formic acid in water
   B: acetonitrile
Flow rate: 0.35 mL/minute
Run time: 3.0 minutes
Flow: Gradient
Column: Acquity UPLC BEH C18 1.7 mm, 2.1 mm×50 mm
Column temp: 50° C.
Sample temp: 10° C.
Injection vol: 2 μL
Weak wash: 1:1:2 CAN:MeOH:water+0.1% formic acid
Strong wash: 1:1:1:1 CAN:MeOH:IPA:water+0.1% formic acid
Wash vols: weak: 800 μL; strong: 400 μL
   The mass spectrometer parameters were as follows. See also Table 7, below.
Capillary voltage: 3. kV
Desolvation temp: 400° C.
Desolvation gas flow: 1000 L/hour
Cone gas flow: 40 L/hour
Source temp: 150° C.

TABLE 7
Mass Spectrometry

| | Ion transition | Cone | Collision |
|---|---|---|---|
| Compounds | (m/z) | (V) | energy (V) |
| Atheliapyrrolidine A | 275.05 > 203.94 | 12 | 18 |
| (quantifier) | | | |
| Atheliapyrrolidine A | 275.05 > 146.89 | 12 | 28 |
| (qualifier) | | | |
| Phenacetin (IS) | 180.12 > 110.03 | 20 | 20 |

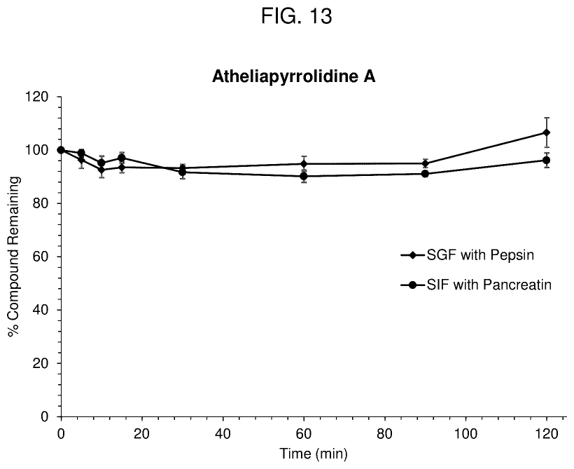

See results in FIG. 13. Atheliapyrrolidine A was found to be stable for 2 hours in both SGF and SIF with enzymes.

### Example 12: Metabolic Stability

The metabolic stability of atheliapyrrolidine A (1 μM) was determined using male CD1 mice liver microsomes (MLM) and mixed gender human liver microsomes (HLM) (1 mg protein/mL). Atheliapyrrolidine A was incubated with MLM and HLM in an incubator/shaker at 37.0° C. and 125 RPM for 5 minutes (N=3 each). The metabolic reaction was started adding 1 mM nicotinamide adenine dinucleotide phosphate (NADPH). Twenty microliter samples were withdrawn at 0, 5, 10, 15, 30, and 60 minutes and mixed with 100 μL of methanol containing 0.05% formic acid and phenacetin (20 ng/ml) as internal standard followed by filtration centrifugation for 5 minutes at 2000 rpm using Solvinert 0.45 μm PTFE filter plates. Verapamil (1 μM) was used as the reference standard for microsomal activity assessment. Two microliters of the filtrate were injected to UPLC-MS/MS for analysis.

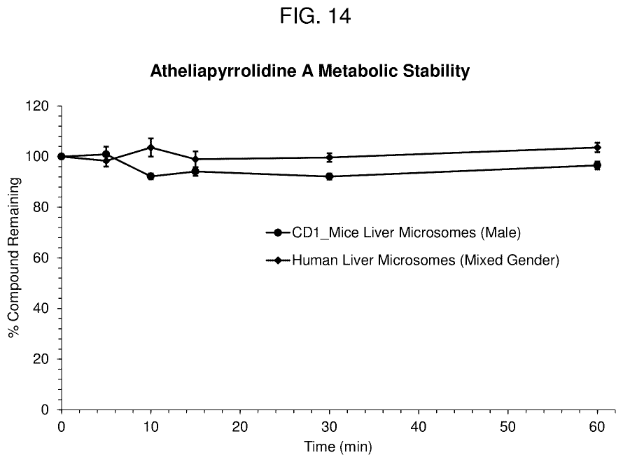

See results in FIG. 14. The half-life of verapamil was found to be 4.5±0.1 and 14.7±0.6 minutes in MLM and HLM, respectively indicating the activity of microsomes during study. Atheliapyrrolidine A was found to be stable for 1 hour in MLM and HLM with less than 10% degradation and the half-life was more than 60 min in both MLM and HLM.

### Example 13: In Vivo Studies in Mice

Male C57BL/6 mice (n=3) were administered a dose of 5 mg/kg of atheliapyrrolidine A, intraperitoneally. The formulation contained 5 mg/3 mL of atheliapyrrolidine A in 10% ethanol, 30% propylene glycol, 5% tween 80, and 55% normal saline. At 0.5 hours post-dose, mouse plasma and brain homogenate samples were taken and analyzed by UPLC-MS/MS. See Example 11 for methods.

The mean plasma and brain concentrations after 30 minutes of I.P. dosing were 1306±342.8 ng/ml and 55.2±12.9 ng/g, respectively. The brain to plasma concentration ratio was found to be 0.04±0.00, showing very limited exposure to brain compared to plasma.

### REFERENCES

All publications listed below and throughout the specification are hereby incorporated by reference in their entirety. Nothing herein is to be construed as an admission that the invention is not entitled to antedate such disclosure by virtue of prior invention.
1. Barnea, G.; Strapps, W.; Herrada, G.; Berman, Y.; Ong, J.; Kloss, B.; Axel, R.; Lee, K. J., The genetic design of signaling cascades to record receptor activation. Proceedings of the National Academy of Sciences 2008, 105 (1), 64-69.
2. da Costa, R. R.; Vreeburg, S. M. E.; Shik, J. Z.; Aanen, D. K.; Poulsen, M., Can interaction specificity in the fungus-farming termite symbiosis be explained by nutritional requirements of the fungal crop? Fungal Ecology 2019, 38, 54-61.
3. de Meiras-Ottoni, A.; Larsson, K.-H.; Gibertoni, T. B., Additions to Trechispora and the status of Scytinopogon (Trechisporales, Basidiomycota). Mycological Progress 2021, 20 (2), 203-222.
4. Gonzalez-Vera, J. A.; Medina, R. A.; Martin-Fontecha, M.; Gonzalez, A.; de la Fuente, T; Vázquez-Villa, H.; García-Cárceles, J.; Botta, J.; McCormick, P. J.; Benhamú, B.; Pardo, L.; and López-Rodríguez, M. L., A new serotonin 5-HT6 receptor antagonist with procognitive activity—Importance of a halogen bond interaction to stabilize the binding. Scientific Reports 2016, 7:41293.
5. Komagata, Y.; Fukasawa, Y.; Matsuura, K., Low temperature enhances the ability of the termite-egg-mimicking fungus Athelia termitophila to compete against wood-decaying fungi. Fungal Ecology 2022, 60, 101178.
6. Konkel, Z.; Scott, K.; Slot Jason, C., Draft Genome Sequence of the Termite-Associated “Cuckoo Fungus,” Athelia (Fibularhizoctonia) sp. TMB Strain TB5. Microbiology Resource Announcements 2021, 10 (1), e01230-20.
7. Kroeze, W. K.; Sassano, M. F.; Huang, X.-P.; Lansu, K.; McCorvy, J. D.; Giguère, P. M.; Sciaky, N.; Roth, B. L., PRESTO-Tango as an open-source resource for interrogation of the druggable human GPCRome. Nature structural & molecular biology 2015, 22 (5), 362-369.
8. ADDIN EN.REFLIST Maekawa, N.; Yokoi, H.; Sotome, K.; Matsuura, K.; Tanaka, C.; Endo, N.; Nakagiri, A.; Ushijima, S., Athelia termitophila sp. nov. is the teleomorph of the termite ball fungus Fibularhizoctonia sp. Mycoscience 2020, 61 (6), 323-330.
9. Matsuura, K.; Tanaka, C.; Nishida, T., Symbiosis of a termite and a sclerotium-forming fungus: Sclerotia mimic termite eggs. Ecological Research 2000, 15 (4), 405-414.
10. Matsuura, K., Distribution of termite egg-mimicking fungi (“termite balls”) in Reticulitermes spp. (Isoptera: Rhinotermitidae) nests in Japan and the United States. Applied Entomology and Zoology 2005, 40 (1), 53-61.
11. Matsuura, K.; Tamura, T.; Kobayashi, N.; Yashiro, T.; Tatsumi, S., The antibacterial protein lysozyme identified as the termite egg recognition pheromone. PLOS One 2007, 2 (8), e813.
12. Matsuura, K.; Yashiro, T.; Shimizu, K.; Tatsumi, S.; Tamura, T., Cuckoo fungus mimics termite eggs by producing the cellulose-digesting enzyme beta-glucosidase. Curr Biol 2009, 19 (1), 30-6.
13. Matsuura, K.; Yashiro, T., Parallel evolution of termite-egg mimicry by sclerotium-forming fungi in distant termite groups. Biological Journal of the Linnean Society 2010, 100 (3), 531-537.
14. Matsuura, K., Termite-egg mimicry by a sclerotium-forming fungus. Proc Biol Sci 2006, 273 (1591), 1203-9.
15. Mitaka, Y.; Matsuyama, S.; Mizumoto, N.; Matsuura, K.; Akino, T., Chemical identification of an aggregation pheromone in the termite Reticulitermes speratus. Sci Rep 2020, 10 (1), 7424.
16. Mitaka, Y.; Mori, N.; Matsuura, K., A termite fungistatic compound, mellein, inhibits entomopathogenic fungi but not egg-mimicking termite ball fungi. Applied Entomology and Zoology 2018, 54 (1), 39-46.
17. Schmidt, S.; Kildgaard, S.; Guo, H.; Beemelmanns, C.; Poulsen, M., The chemical ecology of the fungus-farming termite symbiosis. Natural Product Reports 2022, 39 (2), 231-248.
18. Ye, C.; Li, J.; Ran, Y.; Rasheed, H.; Xing, L.; Su, X., The nest fungus of the lower termite Reticulitermes labralis. Sci Rep 2019, 9 (1), 3384.

1. An atheliapyrrolidine prodrug compound of Formula I: 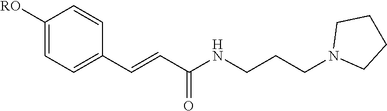
wherein R is a sugar, sugar alcohol, amino sugar, organic acid (e.g. acetic acid), amino acid, 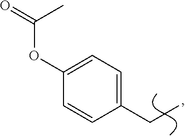
trigonelline, a purine, or a pyrimidine, with the proviso that R is not glucuronic acid;
or a pharmaceutically acceptable salt thereof.

2. A pharmaceutical composition comprising a pharmaceutically acceptable excipient and atheliapyrrolidine A, Atheliapyrrolidine A-GlcA, or the atheliapyrrolidine prodrug compound of claim 1.

3. A method of producing 5-HT_{2A} receptor antagonism in a subject in need thereof, comprising administering a therapeutically effective amount of atheliapyrrolidine A, atheliapyrrolidine A-GlcA, or the atheliapyrrolidine prodrug of claim 1 to the subject.

4. A method of producing 5-HT_{2A} receptor antagonism in a subject in need thereof, comprising administering a therapeutically effective amount of the pharmaceutical composition of claim 2 to the subject.

5. A method of treating a subject in need thereof comprising administering to the subject a therapeutically effective amount of the pharmaceutical composition of claim 2, wherein the subject suffers from a 5-HT_{2A} related disorder.

6. The method of claim 5 wherein the 5-HT_{2A} related disorder is a cardiovascular disorder, a psychiatric or neurological disorder, or pancreatitis.

7. The method of claim 6 wherein the cardiovascular disorder is selected from the group consisting of platelet aggregation, coronary artery disease, myocardial infarction, transient ischemic attacks, stable angina, unstable angina, thrombotic stroke, a secondary ischemic event, atrial fibrillation, and thrombosis.

8. The method of claim 6 wherein the psychiatric or neurological disorder is selected from the group consisting of alcohol and substance use disorders, anxiety disorders, panic disorder, agoraphobia and other specific phobias, social anxiety disorder, post-traumatic stress disorder, obsessive compulsive disorder, generalized anxiety disorder, bipolar disorder, sleep and wake disorders, depression, anorexia nervosa, binge eating disorder, bulimia nervosa, psychosis, schizophrenia, autism spectrum disorders, developmental disorders, and personality disorders.

9. A method of synthesis for the compound of claim 1, or atheliapyrrolidine A comprising:
(a) dissolving trans-p-coumaric acid (1 eq.) in a solvent, wherein the solvent is optionally dry DMF; or other polar, aprotic solvents or mixtures, optionally the solvent comprises DMSO and/or ethyl acetate.
(b) coupling the trans-p-coumaric acid to 1.0 eq. of N-(3-aminopropyl) pyrrolidine using a coupling reagent, optionally EDC*HCl (1.1 eq.) (optionally, DCC, DIC, HATU, CDI, BOPCl) for a sufficient period and temperature to allow reaction, optionally overnight at room temperature;
(c) pre-cleaning the reaction mixture of (b) to remove DMF, optionally by using a solid phase extraction C₁₈ cartridge;
(d) loading the reaction mixture;
(e) washing the cartridge, optionally with H₂O;
(f) eluting the cartridge, optionally with 100% acetonitrile or suitable organic solvent.
(g) evaporating the eluted fraction in vacuo;
(h) resuspending the eluted fraction, optionally in H₂O;
(i) purifying the eluted fraction as a pale yellow powder, optionally by using semi-preparative HPLC-DAD.
